SEQUENCE LISTING (10 sequences)

SEQ ID NO: 1 - DNA, 20 bp, Artificial Sequence
  Synthetic oligonucleotide
cgcaccactggcattgtcat

SEQ ID NO: 2 - DNA, 20 bp, Artificial Sequence
  Synthetic oligonucleotide
ttctccttgatgtcacgcac

SEQ ID NO: 3 - DNA, 19 bp, Artificial Sequence
  Synthetic oligonucleotide
ctggtcgaagcattggaat

SEQ ID NO: 4 - DNA, 21 bp, Artificial Sequence
  Synthetic oligonucleotide
tgtagggcatctgagaacgag

SEQ ID NO: 5 - DNA, 17 bp, Artificial Sequence
  Synthetic oligonucleotide
tcagtggagaccgccca

SEQ ID NO: 6 - DNA, 24 bp, Artificial Sequence
  Synthetic oligonucleotide
tctgaggtctgtcattttctggag

SEQ ID NO: 7 - DNA, 19 bp, Artificial Sequence
  Synthetic oligonucleotide
tgaagagacccaggcgcta

SEQ ID NO: 8 - DNA, 21 bp, Artificial Sequence
  Synthetic oligonucleotide
gatgtggtcagccaactcgtc

SEQ ID NO: 9 - DNA, 21 bp, Artificial Sequence
  Synthetic oligonucleotide
tcaagatcattgctcctcctg

SEQ ID NO: 10 - DNA, 21 bp, Artificial Sequence
  Synthetic oligonucleotide
ctgcttgctgatccacatctg

(19) United States
(12) Patent Application Publication
(10) Pub. No.: US 2012/0128636 A1
(43) Pub. Date: May 24, 2012

(54) Gingiva Derived Stem Cell And Its Application In Immunodulation And Reconstruction

(75) Inventors: Anh D. Le, La Mirada, CA (US); Qunzhou Zhang, South Pasadena, CA (US); Songtao Shi, Irvine, CA (US)

(73) Assignee: UNIVERSITY OF SOUTHERN CALIFORNIA, Los Angeles, CA (US)

(21) Appl. No.: 13/145,541

(22) PCT Filed: Jan. 20, 2010
(86) PCT No.: PCT/US2010/021531

§ 371 Date: Feb. 1, 2012

Related U.S. Application Data
(60) Provisional application No. 61/145,837, filed on Jan. 20, 2009.
(60) Provisional application No. 61/246,066, filed on Sep. 25, 2009.

(51) Int. Cl. A61K 35/12; A61P 1/00; A61P 3/10; A61P 19/02; C12N 5/0775; A61P 29/00

(52) U.S. Cl. 424/93.7; 435/325

(57) ABSTRACT

The present invention relates to gingiva derived messenchymal stem cells (GMSCs). More specifically, the invention provides compositions and methods of using GMSCs to regulate inflammatory response in the setting of normal versus pathological wound healing and to treat inflammatory and/or autoimmune diseases.

The present application claims the benefit of the filing date of U.S. Provisional Application No. 61/145,837 filed Jan. 20, 2009 and 61/246,066 filed Sep. 25, 2009, the disclosure of which is incorporated herein by reference in its entirety.

### STATEMENT REGARDING FEDERALLY SPONSORED RESEARCH OR DEVELOPMENT

This invention was made with government support under Contract No. DE 019932 awarded by the National Institutes of Health. The government has certain rights in the invention.

### FIELD OF THE INVENTION

The invention relates in general to messenchymal stem cell therapy. More particularly, the invention relates to the isolation and application of gingiva derived messenchymal stem cells.

### BACKGROUND OF THE INVENTION

Messenchymal stem cells (MSCs) are multipotent stem cells that can differentiate into a variety of cell types. Cell types that MSCs have been shown to differentiate into in vitro or in vivo include osteoblasts, chondrocytes, myocytes, adipocytes, endotheliums, and beta-pancreatic islets cells.

Mesenchymal stem cells are characterized morphologically by a small cell body with a few cell processes that are long and thin. The cell body contains a large, round nucleus with a prominent nucleolus which is surrounded by finely dispersed chromatin particles, giving the nucleus a clear appearance. The remainder of the cell body contains a small amount of Golgi apparatus, rough endoplasmic reticulum, mitochondria, and polyribosomes. The cells, which are long and thin, are widely dispersed and the adjacent extracellular matrix is populated by a few reticular fibrils but is devoid of the other types of collagen fibrils.

There is currently no test that can be performed on a single cell to determine whether that cell is an MSC. There are surface antigens that can be used to isolate a population of cells that have similar self-renewal and differentiation capacities, yet MSCs, as a population, typically do not all express the proposed markers; and it is not certain which ones must be expressed in order for that cell to be classified as an MSC. It may be that the therapeutic properties attributed to MSCs result from the interaction between the different cells that make up an MSC culture, suggesting that there is no one cell that has all the properties.

MSCs have a large capacity for self-renewal while maintaining their multipotency. Beyond that, there is little that can be definitively said. The standard test to confirm multipotency is differentiation of the cells into osteoblasts, adipocytes, and chondrocytes as well as myocytes and possibly neuron-like cells. However, the degree to which the culture will differentiate varies among individuals and how differentiation is induced, e.g. chemical vs. mechanical; and it is not clear whether this variation is due to a different amount of “true” progenitor cells in the culture or variable differentiation capacities of individuals' progenitors. The capacity of cells to proliferate and differentiate is known to decrease with the age of the donor, as well as the time in culture. Likewise, whether this is due to a decrease in the number of MSCs or a change to the existing MSCs is not known.

Numerous studies have demonstrated that human MSC avoid allorecognition, interfere with dendritic cell and T-cell function and generate a local immunosuppressive microenvironment by secreting cytokines. It has also been shown that the immunomodulatory function of human MSC is enhanced when the cells are exposed to an inflammatory environment characterised by the presence of elevated local interferon-gamma levels. Other studies contradict some of these findings, reflecting both the highly heterogeneous nature of MSC isolates and the considerable differences between isolates generated by the many different methods under development.

The mesenchymal stem cells can be activated and mobilized if needed. However, the efficiency is very low. For instance, damage to muscles heals very slowly. However, if there were a method of activating the mesenchymal stem cells then such wounds would heal much faster. Direct injection or placement of cells into a site in need of repair may the preferred method of treatment, as vascular delivery suffers from a “pulmonary first pass effect” where intravenous injected cells are sequestered in the lungs. Clinical case reports in orthopedic applications have been published, though the number of patients treated is small and these methods still lack rigorous study demonstrating effectiveness. Wakitani has published a small case series of nine defects in five knees involving surgical transplantation of mesenchymal stem cells with coverage of the treated chondral defects.

Although messenchymal stem cells hold great promise for numerous medical applications, up until now, most stem cell therapies are based on well-characterized MSCs derived from bone marrows. Given that extracting stem cells from bone marrows is a difficult procedure with limited yield, this has placed a significant limitation on the development of their therapeutic applications. Recently, adipose stem cells have been investigated as a potential source of stem cells. However, while it is easier to extract adipose stem cells than bone marrow stem cells, the extraction process is still not yet perfected and the resulting stem cells are only suitable for a limited range of applications.

Therefore, there still exists a need for other sources of messenchymal stem cells and new approaches for isolating thereof.

### SUMMARY OF THE INVENTION

In one embodiment, the invention relates to messenchymal stem cells that are derived from gingivia.

In another embodiment, the invention relates to methods for isolating gingiva derived messenchymal stem cells (GMSCs).

In yet another embodiment, the invention relates to methods of using gingiva derived messenchymal stem cells to regulate inflammation in the setting of wound healing or to treat inflammatory and autoimmune diseases, including, but not limited to graft-versus-host disease (GvHD), diabetes, rheumatoid arthritis (RA), autoimmune encephalomyelitis, systemic lupus erythematosus (SLE), multiple sclerosis (MS), periodontitis, intestinal and bowel disease (IBD), alimentary tract mucositis induced by chemo- or radiotherapy, and sepsis.

The GMSCs provided herein possess unique immunomodulatory and anti-inflammatory properties; they exhibit clonogenicity, self-renewal and multi-potent differentiation capacities. Their immunomodulatory capabilities are capable of suppressing peripheral blood lymphocyte proliferation, inducing the expression of a wide panel of immunosuppressive factors including interleukin 10 (IL-10), indoleamine 2,3-dioxygenase (IDO), inducible nitric oxide synthase (iNOS), and cyclooxygenase-2 (COX-2) in response to the inflammatory cytokine, interferon-γ (IFN-γ). They are easy to isolate and they have an abundant tissue source. More importantly, their rapid ex vivo expansion render them an ideal source for stem cell-based therapeutic applications. Exemplary therapeutic methods maybe by either systemic infusion, localized application, or other suitable means of formulation and delivery.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE DRAWINGS

FIG. 1 shows exemplary images of the expression of stem cell markers in human gingival tissues. (A) H & E staining of paraffin-sections of human gingival tissues. MBV, microvascular blood vessel; BV, blood vessel. (B) Frozen sections were immunostained with mouse monoclonal antibodies specific for human Oct-4, SSEA-4 and Stro-1 or an isotype-matched mouse IgG, followed by incubation with FITC-conjugated secondary antibodies. Images were observed under a fluorescence microscope. Scale bar: 100 μm. The results were representative of at least five independent experiments.

FIG. 2 shows exemplary data for the isolation and subcloning of mesenchymal stem cells from human gingival tissues. (A) Subcloning and culture of MSCs from gingival tissues in \grave{α}-MEM supplemented with 10% FBS, 1×NEAA (non-essential amino acid) and antibiotics. Scale bar: 100 μm. (B) Capability of colony formation of gingiva-derived cells. (C) Population doublings of GMSCs. (D) Expression of stem cell markers in GMSCs. Cells cultured in an 8-well slide chamber were fixed and immunostained with specific antibodies for human Stro-1, SSEA-4, Oct-4 or hTERT. Cells were incubated with rhodamine- or FITC-conjugated secondary antibodies and then observed under a fluorescence microscope. Scale bar: 100 μm. (E) Semi-quantification of positive signals in at least 5 random high-power fields and expressed as the percentage of total DAPI-positive cells (mean±SD). (F) Expression of cell surface markers on GMSCs as determined by flow cytometry. (G) Quantification of percentage of cells expressing respective surface markers from independent experiments from flow cytometry data (mean±SD). The results were representative of at least five independent experiments.

FIG. 3 shows exemplary data illustrating the multipotent differentiation of GMSCs. (A) Adipogenic differentiation of GMSCs. After cultured under normal growth condition (control, 1 and 3) or adipogenic differentiation (2 and 4) condition for 2 weeks, adipocyte differentiation was determined by Oil Red O staining and RT-PCR analysis of specific genes. The graph shows the quantification of the Oil Red O dye content in differentiated adipocytes from independent experiments (mean±SD). PPARγ2, peroxisome proliferator activated receptor γ-2; LPL, lipoprotein lipase. (B) Osteogenic differentiation of GMSCs. After cultured under normal growth condition (control, 1 and 3) or osteogenic differentiation (2 and 4) condition for 4-5 weeks, osteogenic differentiation was determined by Alizarin Red S staining and RT-PCR analysis of specific genes. The graph shows the quantification of the Alizarin Red S dye content in differentiated osteocytes from independent experiments (mean±SD). OCN, osteocalcin. Scale bar: 50 μm. (C) Endothelial differentiation of GMSCs after cultured in endothelial cell culture condition for 7 days. Cells were immunostained with a mouse monoclonal IgG for human CD31, followed by incubation with FITC-conjugated secondary antibody, and then observed under a fluorescence microscope. Scale bar: 100 μm. (D) Neural differentiation of GMSCs after cultured in neural cell culture condition for 14 days. Cells were immunostained with different primary antibodies for neural markers, including GFAP, NF-M and \grave{α}-tubulin III, followed by incubation with rhodamine- or FITC-conjugated secondary antibodies, and then observed under a fluorescence microscope. Scale bar: 100 μm. (E) In vivo transplantation of GMSCs. Approximately 2.0×10⁶ stem cells mixed with 40 mg of hydroxyapatite/tricalcium phosphate (HA/TCP) ceramic powder were subcutaneously transplanted into the dorsal surface of 8-10-week-old female immunocompromised mice. 4 weeks later, the transplants were harvested and cells were recovered for secondary transplantation. H & E staining was performed for histological examination. The cells of human origin were confirmed by immunostaining with a specific antibody for human mitochondria. Scale bar: 50 μm. (F) Immunohistochemical studies of the expression of human type I collagen and Oct-4 in GMSC-derived transplants. The results were representative of at least three independent experiments.

FIG. 4 shows exemplary data illustrating the inhibitory effects of GMSC on PHA-stimulated PBMC proliferation. (A)-(B) 2×10⁵ PBMCs were cultured alone or co-cultured with increasing numbers of GMSCs or BMSCs under both cell-cell contact (A) and transwell (B) conditions in the presence or absence of 5 μg/ml PHA for 72 h. Afterwards, cell numbers were counted using Cell Counting Kit-8. *P<0.05; **P<0.01; ***P<0.001; ns, no significant difference. ##P<0.05, as compared with transwell. (C)-(D) GMSCs or BMSCs were pretreated for 2 h with 1-MT (1 mM), L-NAME (500 μM), indomethacin (10 μM), or neutralizing antibodies for IL-10 or TGF-β1 (10 μg/ml), followed by co-culturing with the same number of PBMCs (1:1) under both cell-cell contact (C) and transwell (D) conditions in the presence or absence of 5 μg/ml PHA for 72 h. Afterwards, cell numbers were counted using Cell Counting Kit-8. *P<0.05; **P<0.01; ***P<0.001; #P<0.05; ns, no significant difference (mean±SD). The results were representative of at least three independent experiments.

FIG. 5 shows data illustrating IFN-γ induced IDO expression and IL-10 secretion by GMSCs. (A)-(C) GMSCs or BMSCs were stimulated with increasing concentrations of IFN-γ for 24 h. Then the expression of IDO protein was determined by Western blot, while the IDO activity was analyzed by measuring the concentration of kynurine in the conditioned media (A). IFN-γ-induced IL-10 secretion in the supernatants was determined by using ELISA (B), whereas the expression of iNOS and COX-2 in MSCs in response to IFN-γ was determined by Western blot (C). (D)-(F) 2×10⁵ PBMCs were cultured alone or co-cultured with the same number of GMSCs or BMSCs under cell-cell contact condition in the presence or absence of 5 μg/ml PHA for 72 h. Afterward, the concentrations of IFN-γ (D), IL-10 (E) in the supernatants were determined by using ELISA, whereas IDO protein expression and activity were determined by Western blot and kynurine assay, respectively (F). (G)-(H) PBMCs were pretreated for 2 h with different concentrations of specific neutralizing antibody for human IFN-γ (0.5˜10 μg/ml) or an isotype-matched mouse IgG (10 μg/ml), followed by co-culturing with the same number of GMSCs (1:1) under cell-cell contact condition in the presence or absence of 5 μg/ml PHA for 72 h. Then IDO protein expression and activity were determined by Western blot and kynurine assay, respectively (G), whereas the concentration of IL-10 in the supernatants was determined by using ELISA (H). *P<0.05; **P<0.01; ***P<0.001; ns, no significant difference (mean±SD). The results were representative of at least three independent experiments.

FIG. 6 shows exemplary data illustrating that treatment with GMSCs ameliorates DSS-induced experimental colitis in C57BL/6 mice. Colitis was induced by oral administration of 3% DSS in drinking water for 7 days. 2×10⁶ of GMSCs or BMSCs in 2000 PBS were intraperitoneally injected into mice one day after initiation of DSS treatment. Mice without any treatment (naïve mice) or mice received 200 μl PBS served as controls. At day 10, mice were sacrificed. (A)-(B) Clinical progression of the disease was monitored by body weight changes (A) and colitis score evaluation (B). (C) Colonic MPO activity assays. (D)-(E) Histopathological analysis of colitis. IF, inflammation. Scale bar, 200 μm. *P<0.05; **P<0.01; ***P<0.001; ns, no significant difference (mean±SD). The results were representative of at least three independent experiments.

FIG. 7 shows data illustrating GMSC treatment attenuates colonic inflammatory responses but induces Treg responses in DSS-induced experimental colitis in C57BL/6 mice. Colitis was induced by oral administration of 3% DSS in drinking water for 7 days. 2×10⁶ of GMSCs or BMSCs in 20 μl PBS were intraperitoneally injected into mice one day after initiation of DSS treatment. Mice without any treatment (naïve mice) or mice received 2000 PBS served as controls. At day 10, mice were sacrificed. (A) Immunofluorescence staining and Western blot analysis of the infiltrated CD4+ T lymphocytes in inflamed colons. (B) (E) Immunofluorescence staining and ELISA assays of IFN-γ, IL-17, IL-6 and IL-10 in inflamed colons. (F) Immunofluorescence staining and Western blot analysis of FoxP3 in inflamed colon tissues. Scale bars, 100 μm. *P<0.05; **P<0.01; ***P<0.001; ns, no significant difference (means±SD). The results were representative of at least three independent experiments.

FIG. 8 shows reduction of mucositis in 5-FU induced mucositis mouse models after treatment with GMSCs.

FIG. 9 shows the effects of GMSCs on wound closure in C57BL/6 mice. One day after excisional wound, GMSCs (2×106) were systemically infused by tail vein (i.v.) into mice and wound closure was daily observed. (A) Representative photographs of wounds at different time post-wounding with or without GMSC treatment. (B) Measurement of wound closure at different time points. Scale bars, 100 μm. *P<0.05; **P<0.01.

FIG. 10 shows that GMSCs are home to the injury sites and interact with macrophages. (A) GMSCs prelabeled with CM-DiI were systemically infused by tail vein (i.v.) into mice one day after skin wounding. 7 days after cell injection, skin tissues were frozen sectioned and observed under a fluorescence microscope, whereby normal skin on the other side of the same mice were used as controls. (B) frozen sections of wounded skins from mice after injection with CM-DiI pre-labeled GMSCs were immunostained with FITC-conjugated antibody for mice CD11b. Scale bars, 50 μm.

FIG. 11 shows the anti-inflammatory functions of GMSCs. One day after wound, GMSCs (2×106) were injected via tail vein into mice. At different time points, injured skin was collected and tissue lysates were prepared for MPO activity assay (A) and ELISA assay on inflammatory cytokines, including TNF-α(B), IL-6 (C) and anti-inflammatory cytokine IL-10 (D). *P<0.05; **P<0.01; ***P<0.001

FIG. 12 shows that GMSCs promote the formation of alternatively activated macrophages (AAM) at the wounded sites. (A) Paraffin-embedded sections of wounded skin after injection of GMSCs for 7 days were immunostained with specific antibodies for macrophages (F4180) and alternatively activated macrophages (Arginase-1), showing increased numbers of AAM in the healed wound site after treatment with GMSCs as compared with no cell treatment. Scale bars, 50 μm. (B) The increase in AAM formation in response to GMSC treatment was confirmed by Western blot analysis of arginase-1 (Arg-1) and RELM-a expression by AAM. (C) GMSCs induced AAM formation in a time-dependent manner. *P<0.05; **P<0.01; ***P<0.001

FIG. 13 shows that GMSCs inhibit wound-stimulated degranuation of mast cells. promote the formation of alternatively activated macrophages (AAM) at the wounded sites. (A) One day (24 h) after wound, GMSCs were injected via I.v. into mice. At different time points, wounded skin samples were collected and paraffin-embedded sections were prepared for toluidine staining of mast cells. Scale bars, 50 μm. (B) Quantification of mast cell numbers. (C) Quantification of degranuated or activated mast cells. The results showed that at day 2 post-injury, the number of mast cells significantly decreased in wound sites of mice without GMSC treatment, while GMSCs treatment maintained mast cells in the wound sites (A and B). At day 4 post injury, the number of degranuated mast cells significantly increased while GMSC treatment led to a decrease in mast cell number (B) as well as stabilization of mast cells (A and C). *P<0.05; **P<0.01; ***P<0.001

### DETAILED DESCRIPTION OF THE INVENTION

Mesenchymal stem cells (MSCs) have the capacity to self-renew and differentiate into different cell lineages, including mesodermal, endodermal and ectodermal cells (1, 2). Originally isolated from bone marrow (3), similar subsets of multipotent MSC have also been identified in skin (4, 5), adipose tissue (6), tendon (7), lung, heart, liver (8), placenta (9), amniotic fluid (10), and umbilical cord blood (11). In addition, several populations of MSC have been identified in various dental tissues (12), including dental pulp stem cells (DPSC) (13, 14), stem cells of human exfoliated deciduous teeth (SHED) (15), periodontal ligament stem cells (PDLSC) (16), dental follicle precursor cells (DFPC) (17, 18), and stem cells from apical papilla (SCAP) (19). Asides from the abilities of self-renewal and multipotent differentiation, MSCs commonly express specific genes for embryonic stem cells, such as Octamer-4 (Oct-4) and stage specific embryonic antigen-4 (SSEA-4) (20, 21), and share a similar expression profile of cell surface molecules, such as Stro-1, SH2 (CD105), 5114 (CD73), CD90, CD146, CD29, but typically lack hematopoietic stem cell (HSC) markers, such as CD34 and CD45 (22). At the functional level, MSCs display chemotactic properties similar to immune cells in response to tissue insult and inflammation, thus exhibiting tropism for the sites of injury (23, 24, 25) via production of anti-inflammatory cytokines, and anti-apoptotic molecules. These unique characteristics of MSC make them attractive candidates for the development of novel allogeneic cell-based therapeutic strategies in harnessing inflammation in the repair or regeneration of a variety of damaged tissues (26).

A growing body of evidence has demonstrated that bone marrow derived MSCs (BMSCs) are non-immunogenic and, more importantly, display profound immuno-modulatory and anti-inflammatory capabilities (25, 27, 28). BMSCs exhibit immuno-modulatory effects via inhibiting the proliferation and function of innate and adaptive immune cells such as natural killer (NK), dendritic cells, T and B lymphocytes, as well as promoting the expansion of CD4+CD25+FoxP3+ regulatory T cells (Tregs), via direct cell-cell contact and/or soluble factors (25, 27-29). To date, several soluble factors either produced constitutively by MSCs or as a result of cross-talk with target immune cells, have been attributed to the immuno-modulatory properties of MSCs, including transforming growth factor (TGF)-β1, hepatocyte growth factor (HGF), interleukin (IL)-10, prostaglandin (PEG)-2, nitric oxide (NO), and indoleamine-2,3-dioxygenase (IDO) (29-34). Interestingly, tumor necrosis factor (TNF)-γ and interferon (IFN)-γ, two important pro-inflammatory cytokines secreted by activated T cells, have been demonstrated to stimulate PGE-2, TGF-β1, HGF, NO, and IDO expression by MSCs (29-34). These findings suggest that TNF-\grave{α} and IFN-γ serve as critical feedback signal molecules in the cross-talk between immune cells and MSCs with potential role in MSC-mediated immunosuppressive activities. Furthermore, the immunomodulatory and anti-inflammatory effects of MSCs have been demonstrated in the treatment of several animal disease models, including graft-versus-host disease (GvHD) (35, 36), diabetes (37), rheumatoid arthritis (RA) (38), autoimmune encephalomyelitis (39, 40), systemic lupus erythematosus (SLE) (41), periodontitis (42), inflammatory bowel disease (IBD) (43), and sepsis (44). These studies have provided convincing evidence that BMSC-based therapy may offer potential anti-inflammatory and immunomodulating effects in the treatment of a variety of inflammatory and autoimmune diseases (45).

Up till now, despite the discovery of several MSCs from a variety of tissue sources, most cell-based therapies were conducted using the well-characterized MSC derived from bone marrow (35-41), and recently, adipose tissue (43, 46). In the present invention, we have unexpectedly discovered a new population of MSCs derived from human gingiva (GMSCs), a tissue which is not only easily accessible from the oral cavity but can often be obtained as a discarded biological sample. The GMSCs discovered herein possesses both stem-cell-like and immunomodulatory properties.

Gingiva is a unique oral tissue attached to the alveolar bone of tooth sockets, recognized as a biological mucosal barrier and a distinct component of the oral mucosal immunity. Wound healing within the gingiva and oral mucosa is characterized by markedly reduced inflammation, rapid re-epithelialization and fetal-like scarless healing, contrary to the common scar formation present in skin (47, 48). Such differences in wound healing between gingival/oral mucosa and skin may be attributed to the unique tolerogenic properties of the oral mucosal/gingival immune network (49). Several studies have isolated and characterized progenitor cells in the dermis of skin (4, 5) and within the epithelium of oral mucosa (50), but to date there is a lack of evidence whether a population of progenitor or stem cells exists in the spinous layer of human gingiva. The unexpected discovery described herein provides an abundant source of mesenchymal stem cells, GMSCs, with unique immunomodulatory functions, in addition to the well-documented self-renewal and multipotent differentiation properties. GMSCs are capable to elicit a potent inhibitory effect on T cell proliferation in response to mitogen stimulation. Mechanistically, GMSCs exert their anti-inflammatory effect, partly via IFN-γ-induced stimulation of IDO expression. We will demonstrate herein the in vivo GMSC-based therapy using an established murine model of inflammatory disease, specifically human inflammatory bowel disease (IBD).

Ulcerative colitis and Crohn's disease are two major forms of chronic inflammatory bowel disease (IBD) characterized by dysfunction of the innate and adaptive immunity, resulting in colonic mucosal injuries to the distal small intestine (51, 52). Several well-established murine models of human IBD (53) have provided useful tools for preclinical studies of therapeutic strategies, particularly stem cell-based therapies (43, 46, 54). In this demonstrative example, we show that GMSC infusion attenuated dextran sulfate sodium (DSS)-induced colitis, restored normal digestive function, and stabilized body weight in the tested animal model.

The present invention provides a newly isolated, heretofore unknown population of mesenchymal stem cell derived from gingival, herein referred to as GMSCs. The GMSCs of the present invention have various desirable properties that are useful in both clinical and research applications. In particular, the GMSCs of the present invention has certain immunomodulatory and anti-inflammatory properties not found in other types of mesenchymal stem cells, thus, they are particularly useful in harnessing and modulating inflammatory responses in hosts for cell-based tissue regenerative therapeutic strategies.

Other uses of the GMSCs of the present invention may include cosmetic injection to reduce wrinkles, soft tissue augmentation and other skin rejuvenation based on their ability to synthesize collagen, or any other applications of stem cells known in the art, but are not limited thereto.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Materials and Methods

### Mice

C57BL/6J mice (male, 8-10 week-old, Jackson Laboratories, Bar Harbor, Me.) and beige nude/nude Xid (III) (female, 8-10 week-old, Harlan) were group-housed at the Animal Facility of University of Southern California (USC) under temperature—(72° F.±3°) and air—(50±20% relative humidity) controlled condition, and allowed unrestricted access to standard diet and tap water. Mice were allowed to acclimate for up to 7 days before inclusion in all experiments. All animal care and experiments were performed under the institutional protocols approved by the Institutional Animal Care and Use Committee (IACUC) at USC (USC #11327 and #10941).

### Progenitor Cell Isolation and Culture

Human tissue samples were collected from clinically healthy gingiva of subjects who had no history of periodontal disease and relatively healthy periodontium. The gingival tissues were obtained as remnant or discarded tissues following routine dental procedures at the School of Dentistry, University of Southern California (USC) and the Outpatient Dental Clinic at Los Angeles County (LAC)-USC Medical Center under the approved Institutional Review Board (IRB) protocol at USC.

Gingival tissues were treated aseptically and incubated overnight at 4° C. with dispase (2 mg/ml; Sigma) to separate the epithelial and lower spinous layer. The tissues were minced into 1-3 mm² fragments and digested at 37° C. for 2 hours in sterile phosphate-buffered solution (PBS) containing 4 mg/ml collagenase IV (Worthington Biochemical Corporation, Lakewood, N.J.). The dissociated cell suspension was filtered through a 70 μm cell strainer (Falcon, Franklin Lakes, N.J.), plated on non-treated 10-cm Petri dishes (VWR Scientific Products, Willard, Ohio) with complete alpha-minimum essential medium (\grave{α}-MEM: Invitrogen) containing 10% fetal bovine serum (FBS: Clontech Laboratories, Inc., Mountain View, Calif.), 100 U/ml penicillin/100 μg/ml streptomycin (Invitrogen), 2 mM L-glutamine, 100 mM non-essential amino acid (NEAA), and 550 μM 2-mercaptoethanol (2-ME; Sigma-Aldrich), and cultured at 37° C. in a humidified tissue-culture incubator with 5% CO2 and 95% O2. After 72 hours, the nonadherent cells were removed. The plastic-adherent confluent cells were passaged with 0.05% trypsin containing EDTA, and continuously subcultured and maintained in the complete growth medium. Cells from second to sixth passages were used in the experiments.

### Colony Forming Unit Fibroblasts (CFU-F) Assay

The CFU-F assay was performed as previously described (55, 56). After isolation of the single cell suspension from human gingival tissues, 2×10⁴ cells/cm² were seeded in 60-mm Petri dishes containing complete \grave{α}-MEM and incubated at 37° C. and 5% CO2. After 2-3 days, nonadherent cells were washed off with PBS, and cells were fed twice a week with fresh medium. After 14 days, colonies were washed twice with PBS, fixed for 5 min with 100% methanol, stained with 1% aqueous crystal violet, and counted under a microscope. A CFU-F was defined as a group of at least 50 cells. The CFU-F assay was repeated in 5 independent experiments.

### Single Cell Cloning

A serial dilution method was used to generate single-cell clonogenic culture. For single-cell culture, 100 μl of the final diluted cell suspension (10 cells/ml) was seeded into each well of a non-coated 96-well tissue culture plate containing 100 μl of culture medium (Falcon) (200 μl/well, 4 plates/donor). The plates were screened for presence of single cell colony while wells contained more than two colonies were excluded from further analysis. Wells containing a single cell were allowed to reach confluence, transferred to 24-well dishes, and further expanded in the complete growth medium (57).

### Population Doubling Assay

Clonal gingival precursor cells at each passage (P2, P5, P10 and P20) were seeded at 1.0×10³ cells in 35-mm dishes in complete growth medium as above for several intervals (0, 2, 4, 6, 8, 10 days). Cells were treated with 0.05% trypsin-EDTA and cell number was determined by hemocytometer. Population doubling time (PDT) was calculated with the formula, PDT=(t−t0)*lg2/lg((N/N0) (N0 and N represent the cell numbers at time t0 and t, respectively). Meanwhile, the accumulated population doublings were determined and calculated according to the standard 3T3 protocol as described previously (58).

### Human Bone Marrow MSC Culture

Human bone marrow aspirates from healthy adult donors (20-35 years of age) were purchased from AllCells LLC (Emeryville, Calif.) and cultured with \grave{α}-MEM supplemented with 10% FBS, 100 μM L-ascorbic acid-2-phosphate, 2 mM L-glutamine, 100 U/ml penicillin and 100 μg/ml streptomycin as reported previously (55, 56).

### Flow Cytometric Analysis

Approximately 5×10⁵ cells at passage 2 or 6th were incubated with specific PE- or FITC-conjugated mouse monoclonal antibodies for human CD45, CD29, CD73, CD90, CD105, CD146 (BD Biosciences), Stro-1 and SSEA-4 (R & D System) or isotype-matched control IgGs (Southern Biotech, Birmingham, Ala.) and subjected to flow cytometric analysis (55, 56) using a Beckman Coulter flow cytometer and FACScan program (BD Biosciences, San Jose, Calif.).

### Multipotent Differentiation of Single Colony-Derived GMSC

Osteogenic differentiation: GMSCs were plated at 5×10⁵ cells/well in 6-well plate in MSC growth medium, allowed to adhere overnight, and replaced with Osteogenic Induction Medium (PT-3002, Cambrex, Charles City, Iowa) supplemented with dexamethasone, L-glutamine, ascorbic acid, and β-glycerophosphate. After 4˜5 weeks, the in vitro mineralization was assayed by Alizarin red S (Sigma-Aldrich) staining and quantified by acetic acid extraction method (59).

Adipogenic differentiation: As described above, GMSCs were plated in adipogenic induction medium supplemented with 10 μM human insulin, 1 μM dexamethasone, 200 μM indomethacin, and 0.5 mM 3-isobutyl-1-methylxanthine (Sigma-Aldrich, St Louis, Mo.). After 2 weeks, Oil Red O staining was performed to detect intracellular lipid vacuoles characteristic of adipocytes, and the dye content was quantified by isopropanol elution (5 min shaking) and spectrophotometry at 510 nm (60).

Neuronal differentiation: GMSCs were plated at 1×10⁴ cells/well in 8-well chamber slides (Nalge Nunc, Rochester, N.Y.) coated with poly-D-lysine/laminin and cultured in DMEM/F12 (3:1) (Invitrogen, Carlsbad, Calif.) supplemented with 10% FBS (Invitrogen), IxN-2 supplement (Gibco), 100 U/ml penicillin and 100 μg/ml streptomycin, 10 ng/ml fibroblast growth factor 2 (FGF-2), 10 ng/ml epidermal growth factor (EGF) (R&D Systems, Minneapolis, Minn., USA) and cultured for 14-21 days (61). In all experiments, medium was changed with 50% of fresh medium every 3-4 days.

Endothelial cell differentiation: GMSCs were plated at 1×10⁴ cells/well in 8-well chamber slides (Nalge Nunc) precoated with fibronectin and cultivated in the presence or absence of endothelial growth medium (EGM-2 SingleQuots; Lonza, Walkersville, Md.) for 7 days (62). Medium was changed every 2 days.

### In Vivo Transplantation

Transplantation studies were carried out using single colony-derived GMSCs isolated from five different donors. Three well-characterized single colony-derived populations of GMSCs from separate donors were transplanted in triplicate (n=3). Approximately 2.0×10⁶ stem cells mixed with 40 mg of hydroxyapatite/tricalcium phosphate (HA/TCP) ceramic powder (Zimmer Inc., Warsaw, Ind.) were transplanted into the subcutaneous dorsal pouches of 8-10-week-old female immunocompromised mice as previously described (55, 56).

### RT-PCR

Total RNA was isolated from gingival tissues or cultured cells undergoing adipogenic and osteogenic differentiation using an RNeasy Mini kit (Qiagen). Adipocyte and osteocyte specific genes were amplified using the One-step RT-PCR Kit (QIAGEN, Valencia, Calif.). The specific primers were described as follows: Oct-4 forward primer 5′-CGCACCACTGGCATTG TCAT-3′ and reverse primer 5′-TTCTCCTTGATGTCACGCAC-3′; LPL forward primer 5′-CTGGTCGAAGCATTGGAAT-3′ and reverse primer 5′-TGTAGGGCATCTGAGA ACGAG-3′; PPARγ2 forward primer 5′-TCAGTGGAGACCGCCCA-3′ and reverse primer 5′-TCTGAGGTCT GTCATTTTCTGGAG-3′; osteocalcin forward primer 5% TGAAGAGACCCAGGCGCTA-3′ and reverse primer 5′-GATGTGGTCAGCCAACTCGTC-3′; □-actin forward primer 5′-TCAAGATCATTGCTCCTCCTG-3′ and reverse 5′-CTGCTTGCTGATCCACATC TG-3′. All primers were synthesized at the Core Facility, Norris Comprehensive Cancer Center, at USC.

### Immunofluorescence Studies

4% paraformaldehyde-fixed cultured cells and paraffin-embedded or frozen sections of gingival tissue samples were immunolabeled with specific primary antibodies followed by FITC- and/or rhodamine-conjugated secondary antibodies (BD Biosciences). The primary antibodies include mouse monoclonal IgG for human Oct-4 (C-10, sc-5279; Santa Cruz), SSEA-4 (R & D Systems), CD31 (BioLegend), \grave{α}-tubulin III and neurofilament (NFL; Sigma); mouse monoclonal IgM for human Stro-1 and hTERT (Novus); rabbit polyclonal IgG for human glial fibrillary acidic protein (GFAP) (Sigma). After the nuclei were counterstained with 4′, 6-diamidino-2-phenylindole (DAPI) the samples were observed under a fluorescence microscope. Isotype-matched control antibodies (Invitrogen) were used as negative controls. For semi-quantification, positive signals in at least 5 random high-power fields (HPF) were visualized, counted and expressed as percentage of total DAPI-positive cells (mean±SD).

### Histology and Immunohistochemical Studies

Gingival tissues or GMSC transplants were fixed with 10% formalin in PBS. For histological study, paraffin-embedded sections were stained with hematoxylin and eosin (H & E). For immunohistochemical studies, the paraffin-embedded sections were incubated with specific primary antibodies for human mitochondria, type I collagen or Oct-4 and detected using the universal immunoperoxidase (HRP) ABC kit (Vector, Burlingame, Calif.). They were counterstained with hematoxylin. Isotype-matched control antibodies (Invitrogen) were used as negative controls.

### Western Blot Analysis

Cells were lysed with buffer containing 50 mM Tris-HCl, pH 7.5, 5 mM EDTA, 150 mM NaCl, 0.5% Triton X-100, 10 mM sodium fluoride, 20 mM β-mercaptoethanol, 250 p. M sodium orthovanadate, 1 mM PMSF and complete protease inhibitor cocktail (Sigma), and incubated at 4° C. for 1 hour. The lysates were ultra-sonicated and centrifuged at 12,000 g for 10 min. Protein concentrations were determined by BCA methods. 50˜100 μg protein was separated on 8%˜10% polyacrylamide-SDS gel and electroblotted onto nitrocellulose membrane (Hybond ECL, Amersham Pharmacia, Piscataway, N.J.). After blocking with TBS/5% nonfat dry milk for 2 hours, the membrane was incubated overnight at 4° C. with antibodies against human IDO, COX-2 or iNOS followed by incubation with a horseradish peroxidase (HRP)-conjugated secondary antibody (1:2000) (Pierce) for 45 minutes at room temperature, and the signals were visualized by enhanced chemiluminescence detection (ECL). As a loading control, the blots were re-probed with a specific antibody against human β-actin (1:5000).

### PBMC Proliferation Assay

Different numbers of human GMSCs or BMSCs (2×10³, 4×10³, 2×10⁴) were plated in triplicates onto 96-well plates in 100 μl complete media (RPMI-1640 medium supplemented with 10% FBS, 2 mM L-glutamine, 50 U/ml penicillin and 50 μg/ml streptomycin) and were allowed to adhere to plate overnight. Human peripheral blood mononuclear cells (PBMCs) (AllCells LLC), resuspended at 2×10⁵/ml, were added to wells (2×10⁴ cells/well in 100 μl volume) containing or lacking MSC in the presence or absence of 5 μg/ml phytohemagglutinin (PHA; Sigma). Co-cultures without PHA were used as controls. After 72 hours, 100 μl of cells from each well were transferred to new 96-well plates with 10 μl of Cell Counting Kit-8 (CCK-8; Dojindo Laboratories). The absorbance at 450 nm was measured with a microplate reader.

Transwell experiments were performed in 24-well transwell plates with 0.4 μm size pore membranes (Corning Costar, Cambridge, Mass.). A total of 2×10⁵ PBMCs were seeded to the upper compartment of the chamber, whereby different numbers of GMSCs or BMSCs (2×10⁴, 4×104, 2×10⁵) were seeded to the lower compartment. Cells were cultured in the presence or absence of 5 μg/ml phytohemagglutinin (PHA; Sigma) for 72 hours and analyzed as described above.

In other experiments, neutralizing antibodies for human IL-10, TGF-β1 or an isotype-matched mAb (R & D Systems; 10 μg/ml) and chemical antagonists for COX-2 (indomethacin, 20 μM; Sigma), iNOS (N-nitro-L-arginine methyl ester, L-NAME, 1 mM; Sigma), and IDO (1-methyl-tryptophan, 1-MT, 500 μM; Sigma) were added into the co-culture. All experiments were performed in triplicate and were repeated at least twice.

### IDO Activity/Kynurenine Assay.

Kynurenine is the product of IDO-dependent catabolism of tryptophan. Therefore, the biological activity of IDO was evaluated by determining the level of kynurenine in GMSC culture in response to IFN-γ (PeproTech Inc., Rocky Hills, N.J.) or co-culture with PBMCs in the presence or absence of 5 μg/ml PHA. 100 μl of conditioned culture supernatant was mixed with 50 μl of 30% trichloroacetic acid (TCA), vortexed, and centrifuged at 10,000 g for 5 min. Afterward, 75 μl of the supernatant was added to an equal volume of Ehrlich reagent (100 mg of ρ-dimethylbenzaldehyde in 5 ml of glacial acetic acid) in a 96-well plate, and incubated at room temperature for 10 min. The absorbance at 492 nm was determined. The concentration of kynurenine was quantified according to a standard curve of defined kynurenine (Sigma) concentration (0-150 μM).

### DSS-Induced Murine Colitis

C57BL/6 mice were randomly divided into the following groups (n=6): 1) Naïve group without any treatment; 2) DSS; 3) DSS with human BMSC treatment; 4) DSS with GMSC treatment. Acute colitis was induced by administering 3% (wt/vol) dextran sulfate sodium (DSS, molecular weight 36,000-50,000 daltons; MP Biochemicals) in drinking water, which was fed ad libitum for 7 days (46, 54). 2×10⁶ of GMSCs or BMSCs resuspended in 200 μl PBS were intraperitoneally injected into mice one day after initiation of DSS treatment. Colitis severity was scored (0 to 4) by evaluating the clinical disease activity through daily monitoring of weight loss, stool consistency/diarrhea and presence of fecal bleeding (46, 54). At day 10 after colitis induction, mice were sacrificed by CO₂ euthanasia, and the entire colon was quickly removed and gently cleared of feces with sterile PBS. For protein extraction and myeloperoxidase (MPO) activity assay, colon segments were rapidly frozen in liquid nitrogen. For histopathological analysis, colon segments were fixed in 10% buffered formalin phosphate, and paraffin-embedded sections were prepared for H & E staining. Histological scores were blindly determined as previously described (54).

The infiltration of neutrophils in the colon was assessed by measuring myeloperoxidase (MPO) activity as described before (46, 54). Briefly, colon specimens were homogenized at 50 mg/ml in phosphate buffer (50 mM, pH 6.0) with 0.5% hexadecyltrimethylamonium bromide. The samples were centrifuged at 11,000 g for 15 min at 4° C. The supernatants were diluted 1:30 with 50 mM phosphate buffer (pH 6.0) containing 0.167 mg/ml o-dianisidine (Sigma) and 0.0005% H2O2 (vol/vol). Changes in absorbance at 450 nm were recorded with a spectrophotometer every 30 seconds over 3 minutes. MPO activity was expressed in units per gram of wet tissues, where 1 unit represents the enzyme activity required to degrade 1 μM H2O2/min/ml at 24° C.

### ELISA

The level of IFN-γ, IL-6 and IL-17 in colon tissue lysates was detected using mice ELISA Ready-SET-Go (eBioscience, San Diego, Calif.), following the manufacturers' instructions.

### Statistical Analysis

All data are expressed as mean±SD from at least three independent experiments. Differences between experimental and control groups were analyzed by two-tailed unpaired Student's t-test using SPSS. P-values less than 0.05 were considered statistically significant.

### Results

Isolation and Characterization of MSC from Human Gingival Tissues

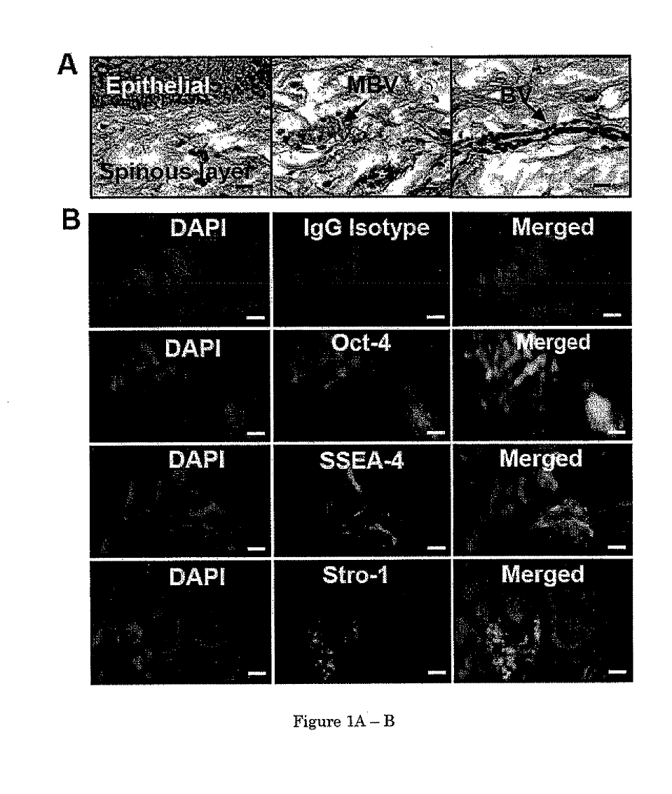

A variety of post-natal or adult stem cells and/or precursor cells have been reported in several complex human tissues or organs (8, 22), including the dental tissues (63); however, to date, no study has confirmed whether such a population of precursor cells exists in human gingiva. Histologically, gingiva is composed of an epithelial layer, a basal layer, and a lower spinous layer that is similar to the dermis of the skin. Here, we demonstrated that human gingival tissues display Octamer-4 (Oct-4), stage specific embryonic antigen-4 (SSEA-4), and Stro-1 positive signals which were clustered in the sub-epithelial connective tissue proper (the lower spinous layer) (FIGS. 1, A and B). Meanwhile, dual-color immunofluorescence studies showed the co-expression of Oct-4/SSEA-4 or Oct-4/Stro-1 by a proportion of cells in gingival tissues (FIG. S1A). In addition, the expression of Oct-4 mRNA in gingival tissues was further confirmed by RT-PCR (FIG. S1B). These results suggest the presence of putative population of stem cells in human gingiva (20, 21).

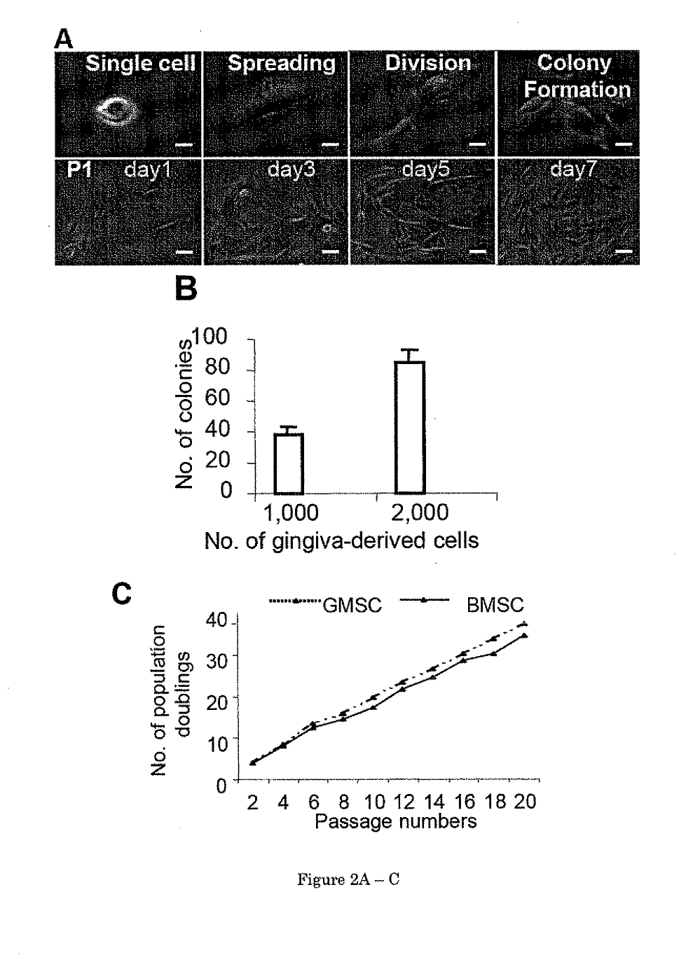

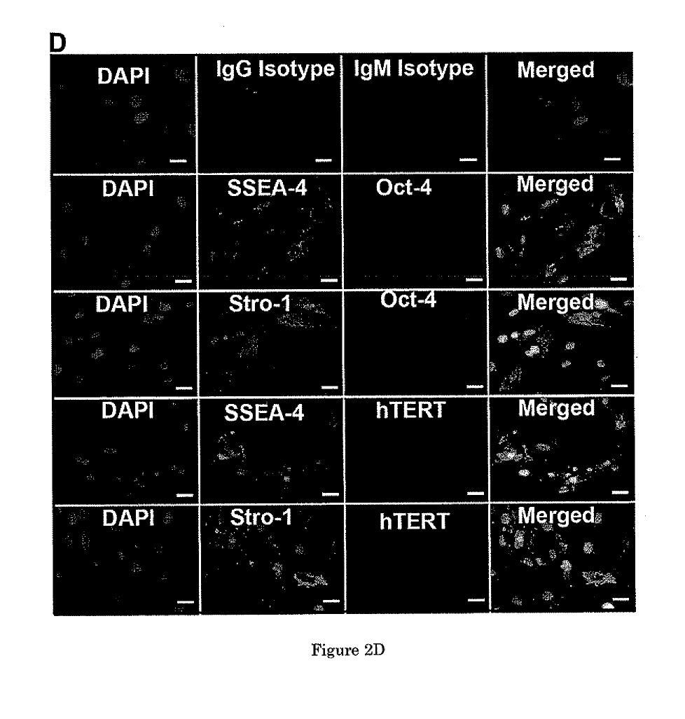

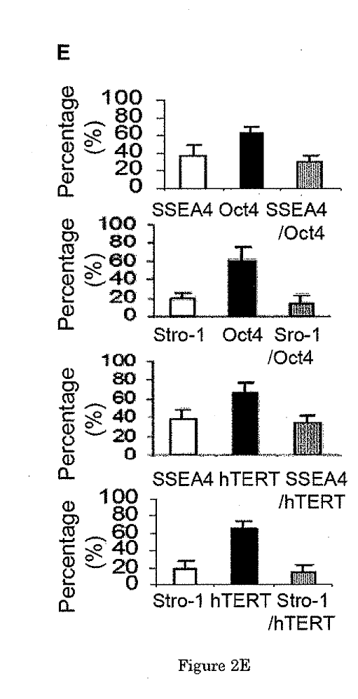

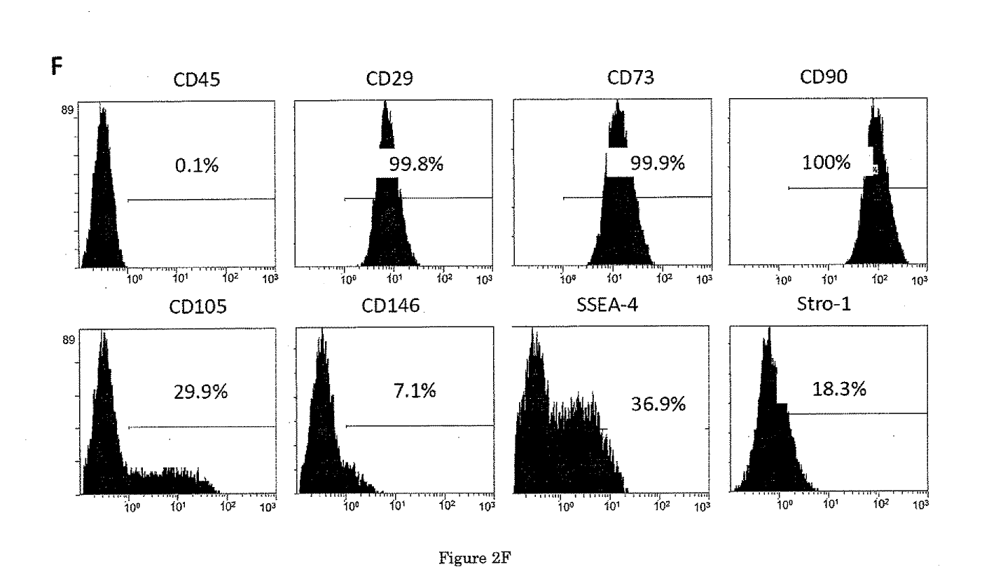

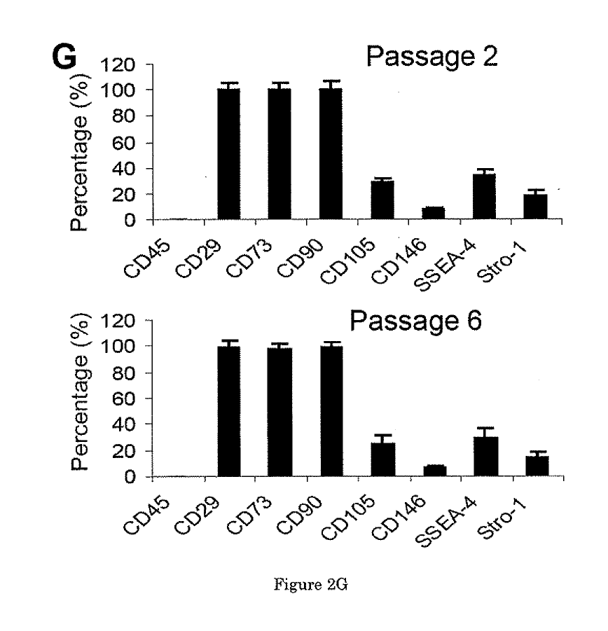

Using normal gingival tissues obtained from 5 healthy donors, we isolated a population of non-epithelial progenitor cells namely human gingiva-derived mesenchymal stem cells (GMSCs), and characterized their stem cell-like properties. Similar to BMSCs, human GMSCs adhered to culture dishes and organized as single colony-forming units (FIG. 2A). Colony formation was observed in approximately 4-6% of GMSCs (FIG. 2B). Like other dental derived stem cells such as PDLSC and SHED (15, 16), GMSCs showed relatively higher proliferation rate and number of population doublings as compared to BMSCs (FIG. 2C). Adherent cells isolated from a small piece of gingival tissue (approximately 2×2 mm²) usually reached confluence (˜1−2×10⁶ cells) after cultured for 10˜14 days (data not shown). Immunocytochemical studies showed that about 60% of single colony-derived GMSCs expressed Oct-4 and human telomerase reverse transcriptase (hTERT), respectively, while 18-20% of cells expressed Stro-1 (FIGS. 2, D and E). Dual-color immunostaining revealed about 30% of GMSCs co-expressed SSEA-4/Oct-4 while ˜15% of cells co-expressed Stro-1/Oct-4 or Stro-1/hTERT (FIGS. 2, D and E), confirming the presence of early mesenchymal progenitor cell phenotype. To further verify the stem cell phenotypic markers of GMSCs using flow cytometry, we observed that ˜100% of GMSCs were negative for CD45, a haematopoietic cell surface marker, but consistently expressed CD29, CD73 and CD90/Thy-1 at passages 2 and 6, while 36.9%, 29.9% and 18.3% of GMSCs were consistently positive for SSEA-4, CD105 and Stro-1, respectively (FIGS. 2, F and G). Most recently, a study reported that BMSCs could be expanded in monolayer up to passage 5 without altering their undifferentiated phenotype (64). Similarly, our results suggested that GMSCs could be steadily expanded in vitro and still maintained their early phenotypes at passage 6. In addition to these stem cell markers, MSCs derived from various human tissues including bone marrow (22), adipose (65), and dermis (5, 65) have also been reported to express extracellular matrix components characteristic of mesenchymal stromal cells, such as vimentin, fibronectin and type I collagen. Consistent with these findings, our in vitro cultured GMSCs also expressed type I collagen as determined by Western blot analysis (FIG. S1C).

### GMSC are Capable of Multiple Differentiation

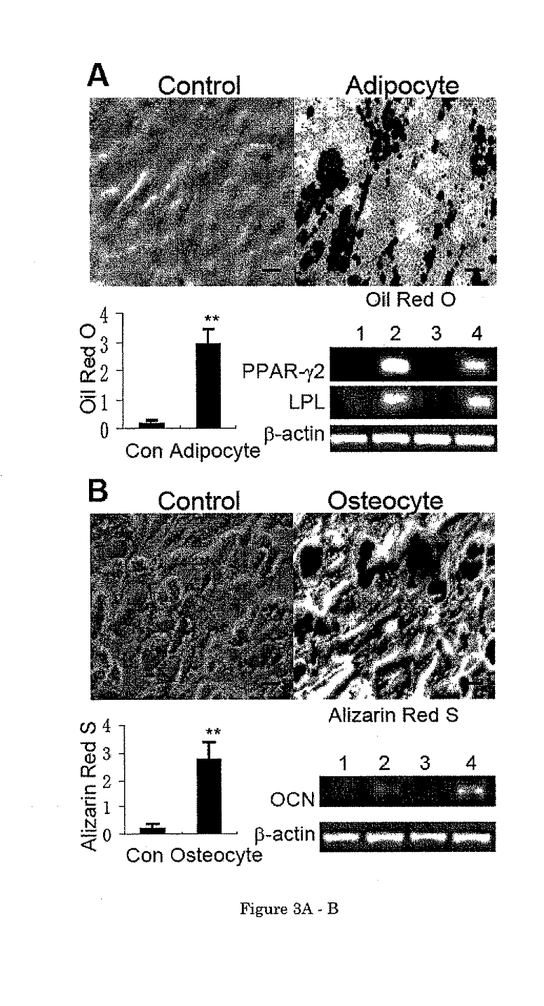

We next examined the multi-differentiation potential of GMSCs. Under adipogenic and osteogenic induction conditions, single colony-derived GMSCs could differentiate into adipocytes and osteoblasts as determined by Oil Red O (FIG. 3A) and by Alizarin Red S staining (FIG. 3B), respectively. Adipogenic differentiation was further confirmed by the increased expression of specific adipogenic markers including peroxisome proliferator-activated receptor γ2 (PPARγ2), lipoprotein lipase (LPL) as determined by RT-PCR (FIG. 3A). Likewise, the osteogenic differentiation of GMSCs was further confirmed by the increased expression of osteocalcin, an osteogenic marker (FIG. 3B). When cultured on fibronectin-coated slide in endothelial cell growth medium for 1 week, about 36.7±8.7% of GMSCs expressed endothelial cell marker CD31, whereas no CD31 positive cells were observed under normal non-induction condition (FIG. 3C; FIG. S2A). Under neural differentiation conditions, about 34.8±9.4%, 22.27±6.5%, and 16.8±5.1% of cells were positive for glial fibrillary acidic protein (GFAP), neurofilament 160/200 (NF-M), and 6-tubulin III, respectively (FIG. 3D; FIG. S2B), whereas no cells positive for these neural markers were detected in normal non-induced medium (FIGS. S2, B and C). These findings, consistent with mesenchymal stem cell properties described in other tissues, indicate that single colony-derived human gingiva stem cells represent a putative MSC population with clonogenic renewal and multipotent differentiation capacities.

To explore the in vivo differentiation capability, the expanded subclonal GMSCs (2×10⁶) were subcutaneously transplanted using hydroxyapatite/tricalcium phosphate (HA/TCP) as a carrier in immunocompromised mice. Similar transplants were carried out using human BMSCs as another source of stem cells. Unlike BMSC transplant, which showed formation of bone nodules in vivo, GMSCs from several donors consistently regenerated connective tissue-like transplants (5 out of 5 mice), with the histological features of early connective tissue phenotype, including presence of collagen fibers (FIG. 3E). The human origin of cellular components of the transplants was confirmed by immunostaining with specific antibodies to human mitochondria (FIG. 3E). Therefore, under our experimental conditions using HA/TCP as carrier, we did not observe osteogenic differentiation in subcutaneous transplants with GMSCs. The fate of in vivo lineage commitment of MSCs depends on multiple factors, such as different tissue origins, the hierarchy of lineage commitment, cell culture conditions, local growth factors, and transplantation conditions (carriers and recipients) (13, 16). Then further studies are warranted to determine whether in vivo osteogenic differentiation of GMSCs can be induced by modifying the transplant carrier and niche components.

To further confirm the renewal and differentiation capability of GMSCs, we performed serial subcutaneous transplantation using HA/TCP carrier and 2×10⁶ GMSCs in immunocompromised mice. At 4 weeks post-primary transplantation, the transplants were harvested and digested single cells were re-transplanted subcutaneously into immunocompromised mice to generate the secondary transplant (FIG. 3E). Our results indicated that GMSCs recovered from primary transplants maintained the expression of Oct-4 and the in vivo ability to self-renew, and formed connective-like tissues expressing type I collagen (FIG. 3F). Together, these results indicated that GMSCs represent a new population of stem cells derived from human gingiva with self-renewal and unique differentiation capabilities.

### GMSC are Capable of Suppressing PBMC Proliferation

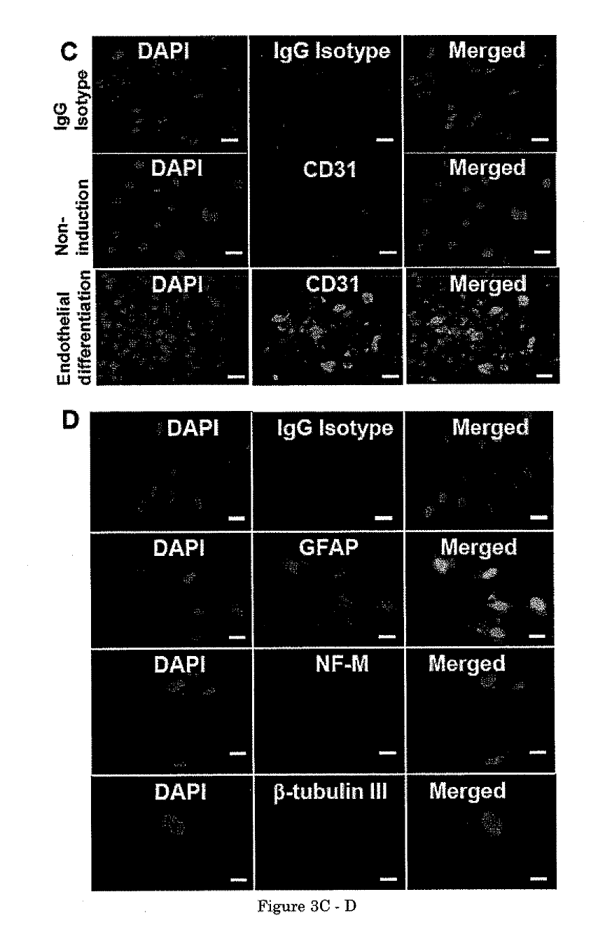

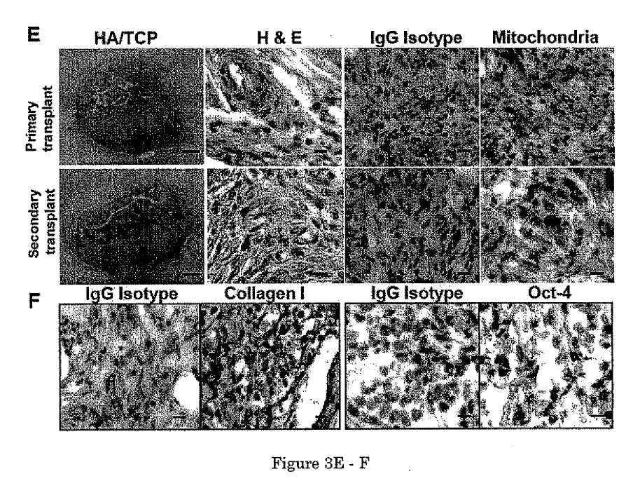

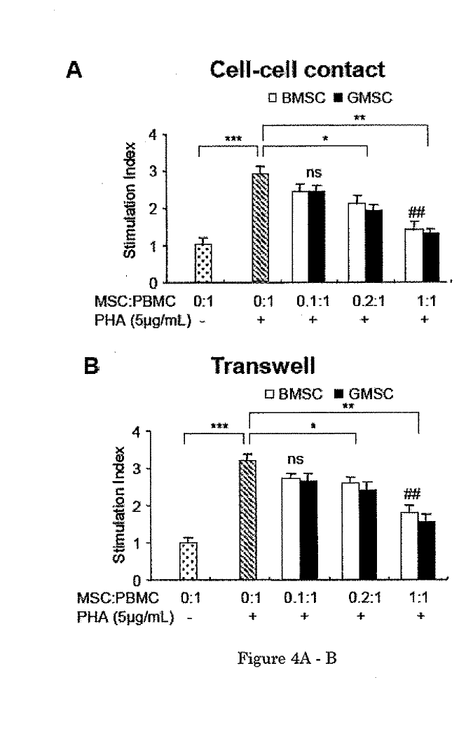

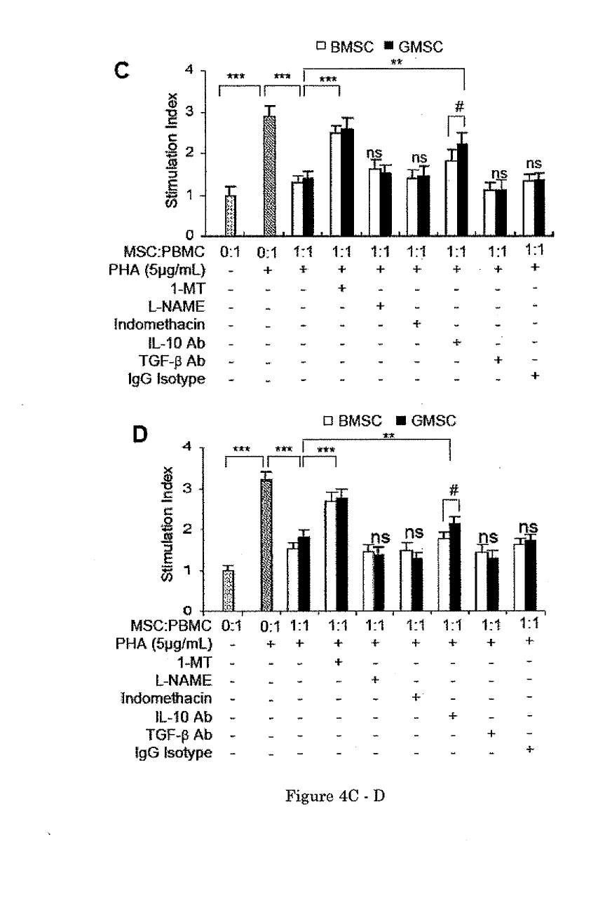

Next, we sought to determine whether GMSCs had immunosuppressive effects on the proliferation of T lymphocytes in response to mitogenic stimulation in vitro. To this end, GMSCs or BMSCs were co-cultured under cell-cell contact or transwell systems with increasing numbers of human peripheral blood mononuclear cells (PBMCs) in the presence of PHA for 72 hours. Our results showed that GMSCs, similar to BMSCs, inhibited mitogen-stimulated PBMC proliferation at a cell density-dependent manner under both cell-cell contact and transwell cultures (FIGS. 4, A and B). Meanwhile, our data also indicated that GMSCs mediated inhibition of PBMC proliferation was more severe under cell-cell contact conditions than in transwells (P<0.05; FIGS. 4, A and B). In addition, the viability of PBMCs co-cultured with GMSCs under both conditions cell-cell contact and transwells, was more than 95%, as determined by trypan blue exclusion (data not shown). These results suggest that direct cell-cell contact contributes, at least in part, to the mechanisms of GMSC-mediated immunosuppression via suppression of PBMC proliferation.

### Soluble Mediators Involved in GMSC-Mediated Inhibition of PBMC

We next determined the role of soluble mediators in GMSC-mediated suppression of PBMC proliferation. To this purpose, GMSCs or BMSCs were pretreated with neutralizing antibodies for human IL-10, TGF-81 or an isotype-matched mAb, or with chemical antagonists for COX-2 (indomethacin), iNOS (1-NAME) or IDO (1-MT) for at least 2 hours, followed by co-culture with PBMC in the presence of PHA stimulation for 72 hours. Our results showed that pretreatment with 1-MT, a specific inhibitor of IDO, significantly reversed GMSC- and BMSC-mediated inhibition of PBMC proliferation under both cell-cell contact and transwell conditions (P<0.001; FIGS. 4, C and D). In addition, treatment with IL-10 neutralizing antibody could reverse the inhibitory effect of PBMC proliferation exerted by GMSC to a better extent than that mediated by BMSCs (P<0.05; FIGS. 4C and 4D). However, treatment with neutralizing antibody for TGF-β1, antagonists for COX-2 and iNOS, or an isotype-matched control IgG, could not reverse the inhibitory effects of GMSCs on PBMC proliferation (FIGS. 4, C and D). These results suggest that IDO and IL-10, not TGF-β1 or COX-2 or iNOS, may contribute, in part, to GMSC-mediated suppression of PBMCs.

Up-regulation of IFN-γ-Induced IDO and IL-10 Contributes to GMSC-Mediated Suppression of PBMC

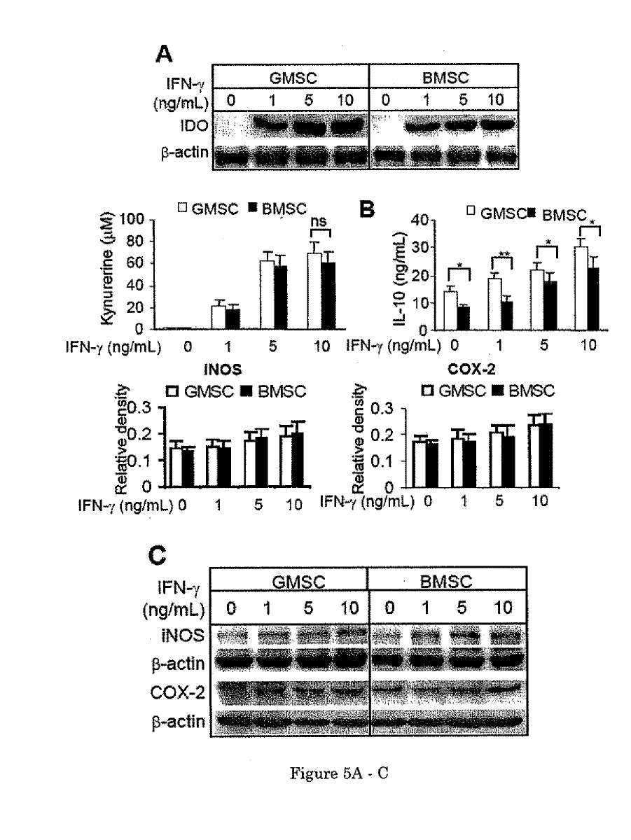

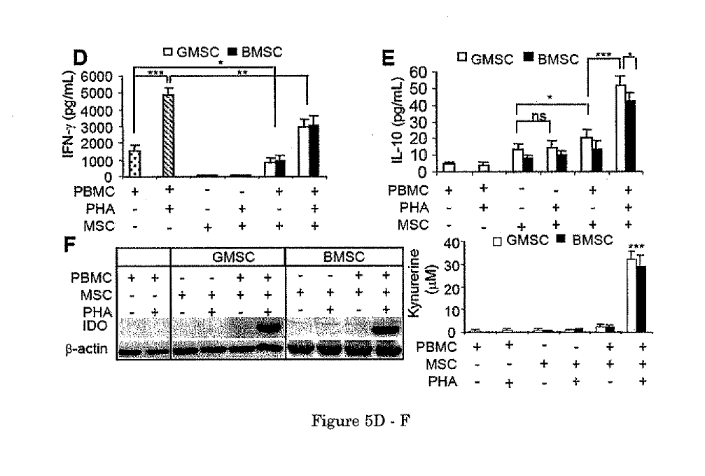

Previous studies have shown that the inflammatory cytokine IFN-γ is capable to regulate the immunomodulatory functions of MSC via up-regulation of a variety of immunosuppressive factors, including IDO and IL-10 (29-34). MSCs have been reported to inhibit the secretion of IFN-γ by PHA-activated immune cells (11, 31, 32). We examined whether IFN-γ could up-regulate IDO and IL-10 expression in GMSCs. Here, we demonstrated that IFN-γ induced IDO protein expression in GMSCs in a dose dependent manner, albeit to a similar extent as in BMSCs (FIG. 5A). Meanwhile, functional assays confirmed that the concentration of kynurenine, a metabolic product of IDO, increased in supernatants of both

GMSCs and BMSCs in response to IFN-γ stimulation (FIG. 5A), suggesting the MSC-induced IDO molecule was active. Moreover, IFN-□ also stimulated IL-10 secretion by GMSC in a dose-dependent manner, an effect stronger than that observed in BMSCs (P<0.05; FIG. 5B). However, only a mild increase in the expression of iNOS and COX-2 was detected in both GMSC and BMSCs in response to IFN-γ (FIG. 5C).

We next determined whether immunosuppressive factors such as IFN-γ, IL-10 and IDO were expressed by PBMCs cultured alone or co-cultured with MSCs in the presence or absence of PHA stimulation. As expected, mitogen stimulation robustly triggered IFN-γ production by PBMCs (P<0.001); however, this burst of IFN-γ was abrogated by co-culture with GMSCs, both at the basal level and in the presence of PHA stimulation (FIG. 5D). We confirmed that PBMCs secreted very low levels of IL-10 either in the absence or presence of PHA, and PHA only had a slight effect on IL-10 production in both GMSCs and BMSCs (P>0.05)(FIG. 5E). However, the secretion of IL-10 by GMSCs was moderately induced in the presence of PBMCs (P<0.05), an effect which was more augmented when co-cultured with PHA-stimulated PBMCs (P<0.001; FIG. 5E), albeit to a greater response than in BMSCs (P<0.05; FIG. 5E). In addition, we determined the expression and activity of IDO under similar conditions. As shown in FIG. 5F, PHA stimulation had no effects on IDO protein expression and activity in both PBMCs and BMSCs. However, in the presence of PITA-stimulated, not the un-stimulated PBMCs, a substantial up-regulation of IDO expression and activity was observed in both GMSCs and BMSCs (FIG. 5F). Taken together, these results suggest that GMSCs upon activation by PHA-stimulated PBMCs are capable of enhanced IL-10 secretion and IDO activity.

Based on these findings, we postulate that the increased IL-10 secretion and IDO expression by GMSCs may be attributed to an increased IFN-γ production by PHA-stimulated PBMCs. We pretreated PBMCs with increasing concentrations of IFN-γ neutralizing antibody followed by co-culture with GMSCs in the presence or absence of PHA for 24 hours. Our results showed that treatment with IFN-γ neutralizing antibody led to a dose-dependent inhibition of IDO expression/activity and IL-10 secretion by GMSCs upon co-culture with PHA-stimulated PBMCs (FIGS. 5, G and H). Similar results were observed in BMSCs, whereas no inhibitory effects were seen in the control group treated with an isotype-matched control antibody (FIGS. 5, G and H). Altogether, these findings support the notion that IFN-□ directly contributes to GMSC-mediated suppression of PBMCs through the up-regulation of IL-10 and IDO expression.

### GMSC-Based Therapy Ameliorates Dss-Induced Colitis in Mice

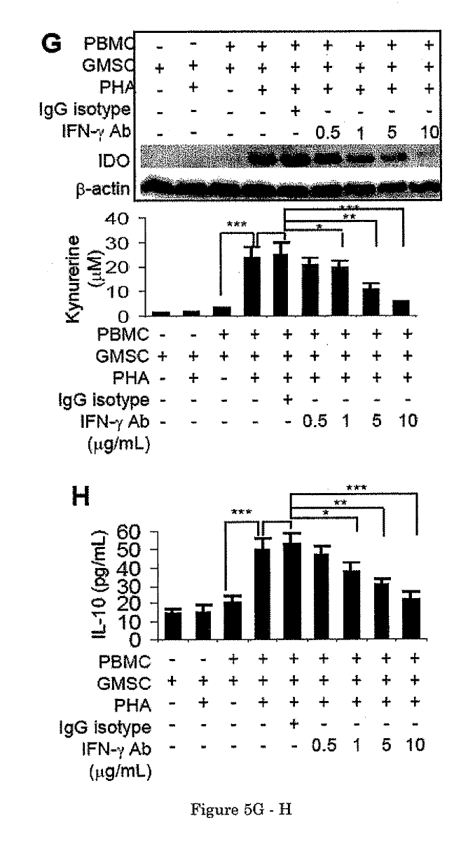

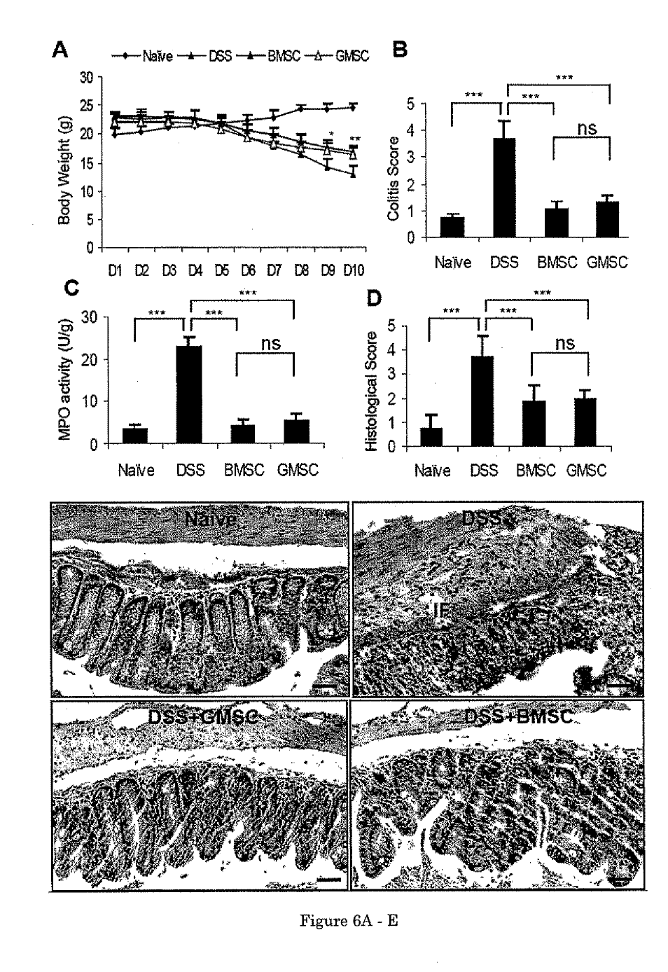

Based on the unique immunomodulatory properties of GMSCs, we next explored the potential therapeutic effects of GMSC infusion in harnessing inflammation and reversing inflammatory-related tissue injuries using an established murine model of colitis induced by oral administration of DSS (54). Similar to previous reports (46, 54), we confirmed that oral administration of 3% DSS for 7 days induced acute colitis in C57BL/6 mice characterized by an overall elevation of colitis scores based on the presence of sustained weight loss and bloody diarrhea/loose feces (FIGS. 6, A and B). Histological studies revealed severe colonic transmural inflammation with increased wall thickness and localized inflammatory cell infiltration, epithelial ulceration with degeneration of crypt architecture, and loss of goblet cells (FIGS. 6, D and E). The histopathological disease activity of induced colitis was assessed by measuring MPO activity released from local neutrophil infiltration (FIG. 6C). Importantly, our results showed that systemic infusion with GMSCs, similar to BMSCs, protected mice against colitis-related tissue injuries and reduced the overall disease severity, shown here as a decrease in disease score, reversing and stabilizing of body weight (P<0.05), suppressing of colonic inflammation (P<0.001; FIGS. 6, A and B), and MPO activities (P<0.001; FIG. 6C). Histologically, GMSCs significantly ameliorated colonic transmural inflammation and decreased wall thickness, restored goblet cells, suppressed mucosal ulceration and focal loss of crypts, thus restored normal intestinal architecture and resulting in a reduced histological colitis score (P<0.001; FIGS. 6, D and E). These compelling findings suggest that cell based therapy using GMSCs can alleviate DSS-induced experimental colitis in mice.

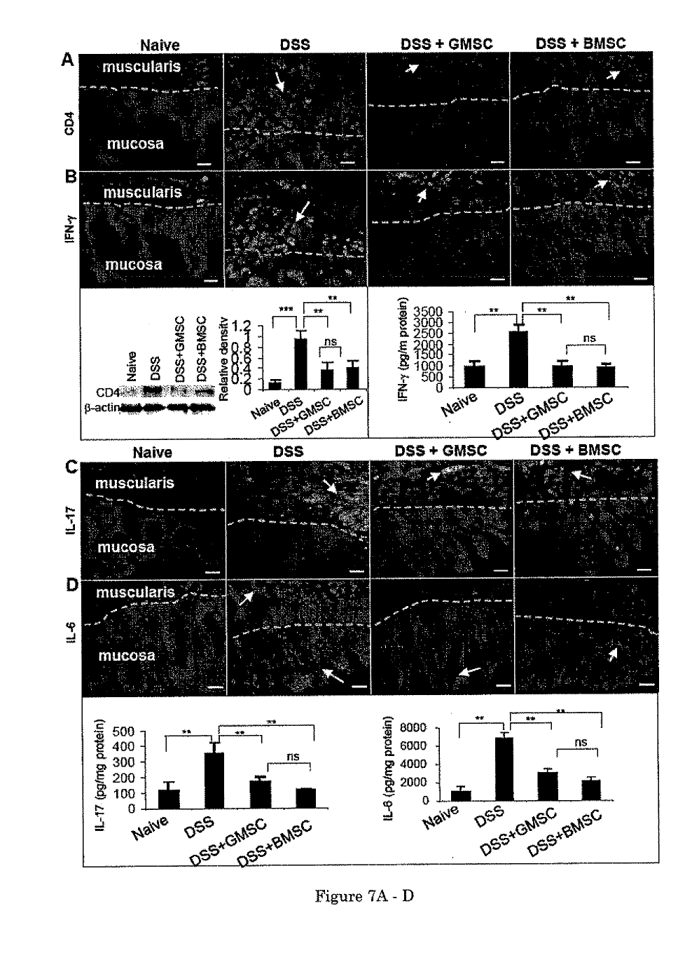

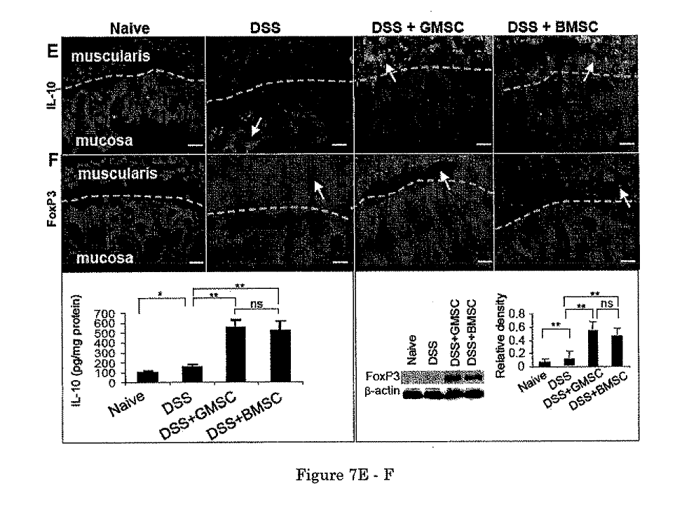

We next investigated the in vivo effects of GMSCs on inflammatory cell response and production of local inflammatory cytokines mechanistically linked to inflammatory-related colonic injuries in DSS-induced colitis (43, 46, 54). We observed an increased infiltration of CD4+ T lymphocytes in the mucosal and muscularis layers of the inflamed colons of colitic mice as determined by immunofluorescence studies and semi-quantified Western blot analysis (P<0.001; FIG. 7A). Meanwhile, immunohistochemical studies showed abundant expression of pro-inflammatory cytokines (IL-6 and IFN-γ) in the mucosal and muscularis layers of the inflamed colons of colitic mice (FIGS. 7, B and D), whereas IL-17 signals were restricted only in the muscularis layer (FIG. 7C). The increased expression of these inflammatory cytokines in inflamed colons was further confirmed and quantified by ELISA (P<0.01; FIGS. 7, B, C and D).

Systemic infusion with GMSCs, similar to BMSCs, significantly attenuated the local recruitment of CD4+ T lymphocytes at the colonic sites (P<0.01; FIG. 7A). The suppression of CD4+ T lymphocytes infiltration elicited by treatment with GMSCs or BMSCs was accompanied by down-regulated levels of inflammatory cytokines, specifically at the intestinal mucosa, with minimal basal activities in the deeper layers (P<0.01; FIGS. 7, B, C and D). Interestingly, systemic infusion of GMSCs, similar to BMSCs, also significantly increased the level of anti-inflammatory cytokine IL-10 and promoted the infiltration of regulatory T cells (Tregs) demonstrated as the expression of the specific transcriptional factor, FoxP3, or by immunostaining, ELISA, and semi-quantitative Western blot analyses (P<0.01; FIGS. 7, E and F). These compelling findings suggest that GMSC treatment confers significant protection against inflammatory-related colonic injuries in experimental colitis by suppressing inflammatory cell infiltration and pro-inflammatory cytokine secretion as well as by increasing the accumulation of Tregs and IL-10 expression at the local intestinal sites.

### Discussion

In the present invention, we have isolated and characterized a new population of precursor cells from human gingival tissues, termed GMSC, which exhibit several unique stem cell-like properties similar to those of MSC derived from bone marrow and other post-natal tissues (8, 22). These characteristics include in vitro proliferation as plastic-adherent cells with fibroblast-like morphology, colony forming ability, multipotent differentiation into different cell lineages including mesodermal (adipocytes, osteocytes), endodermal and neuroectodermal progenies, and expression of mesenchymal cell surface markers and stem cell specific genes (1-3, 20, 21). More importantly, we have demonstrated that single colony-derived GMSCs possess in vivo self-renewal and differentiation capacities, further supporting their stem cell-like properties. In addition, as compared to MSCs derived from several other adult dental tissues such as dental pulp stem cells (DPSC) (13, 14) and periodontal ligament stem cells (PDLSC) (16, 18), GMSCs express a similar profile of cell surface molecules, a high proliferative rate, and an increased population doublings, thus can be easily expanded ex vivo for several cell-based clinical applications. Interestingly, subcutaneous transplantation of GMSCs could form connective tissue-like structures, whereas transplantation of DPSCs and PDLSCs could generate dentin-like and cementum/PDL-like structures (13, 14, 16). These findings have provided evidence that human gingiva, an easily accessible tissue from the oral cavity, or a discarded tissue sample following some dental procedures, might serve a unique source of postnatal stem cells with potential therapeutic functions in tissue regeneration and repair (1-3, 12).

In recent years, a major breakthrough was the discovery that MSCs are immune-privileged and more importantly, possess profound immunomosuppressive and anti-inflammatory effects both in vitro and in vivo via inhibiting the proliferation and function of several major types of innate and adaptive immune cells such as natural killer (NK) cells, dendritic cells, T and B lymphocytes (25, 27-29). However, to date, the underlying mechanisms of MSC-mediated suppression of lymphocyte proliferation remain largely unknown (28, 30, 31, 33, 66, 67). In one study the suppressive activity of human bone marrow MSC was shown to be independent of cell-cell contact (31); however, several other studies have reported that cell-cell contact contributed, at least in part, to the immunosuppression mediated by MSCs derived from human bone marrow, adipose or umbilical cord blood (32, 68, 69). In the present invention, we showed that cell-cell contact may partially contribute to GMSC-mediated suppression of PBMC proliferation. This is evidenced by the observation that when co-cultured with PBMCs under cell-cell contact condition, GMSCs exhibited a slightly stronger inhibition on PBMC proliferation than when co-cultured with PBMCs separately in transwells.

Various studies have indicated that soluble factors such as transforming growth factor (TGF)-61, hepatocyte growth factor (HGF), interleukin (IL)-10, HLA-G5, prostaglandin (PGE-2), nitric oxide (NO), and indoleamine 2,3-dioxygenase (IDO), play an important role in MSC-mediated immunosuppression (27-35, 66-69). However, it is noteworthy that the relative contribution of these soluble factors to the immunosuppressive effects of MSC varies under different experimental conditions, and neutralizing these soluble factors does not completely abrogate the immunosuppressive activity of MSC (32). For example, IL-10, HGF, and TGF-131 have been shown to contribute to BMSC-mediated immunosuppression (33, 66), but in other studies, these three factors appeared not related to immunosuppression mediated by BMSCs and human adipose-derived stem cells (hASCs) (30, 31, 67). In addition, controversies about the role of PGE-2 in MSC-mediated immunosuppression have also been reported. In some studies, blocking PGE-2 production by COX-2 resulted in partial abrogation of mmunosuppression by BMSCs and hASCs (29, 30, 32, 33); however, Cui et al have recently reported that PGE-2 is the major soluble factor in the in vitro inhibition of allogeneic lymphocyte reaction (67). In the present invention, we observed that blocking TGF-61, PGE-2 or NO by using specific neutralizing antibodies or antagonists for synthetic enzymes showed no obvious effects on GMSC-mediated suppression of PBMC proliferation. However, blocking IL-10 led to moderate abrogation of GMSC-mediated suppression of PBMC proliferation, albeit to a greater extent than in BMSCs. These findings suggest that IL-10 might partially contribute to GMSC-mediated immunosuppression.

Indoleamine 2,3-dioxygenase (IDO) is an enzyme that catabolizes tryptophan, an essential amino acid. A growing body of evidence has indicated that IDO plays a critical role in immunosuppression mediated by MSCs of various tissue origins, whereas 1-methyl L-tryptophan (1-MT), a specific antagonist of IDO, can abrogate the immunosuppressive effects (30, 31, 32, 33, 70). The immunomodulatory effects of IDO are attributed to tryptophan depletion and/or accumulation of the downstream metabolites such as kynurenine, 3-hydroxykynurenine, and 3-hydroxyanthranilic acid (30, 31, 32, 33, 70). Most recently, studies have shown that IDO activity is involved in periodontal ligament stem cells (PDLSCs) and gingival fibroblasts (GEO-mediated immunosuppression (70, 71). Consistently, we have demonstrated that the addition of 1-MT also significantly ablated GMSC-mediated suppression of PBMC proliferation in response to mitogen stimulation under both cell-cell contact and transwell conditions, suggesting that IDO might play a major role in GMSC-mediated immunosuppression.

Generally, IDO is not constitutively expressed by mesenchymal stromal cells, but can be significantly induced by a variety of inflammatory mediators (30, 32, 71). Accumulating evidence has shown that IFN-γ plays a critical role in the cross-talk between MSCs and immune cells. Upon activation, immune cells secrete a high amount of inflammatory cytokines, especially IFN-γ, which may subsequently stimulate MSCs to express various immunosuppressive molecules, such as IDO, resulting in a negative feedback inhibition of inflammatory cell responses in terms of proliferation and cytokine secretion (11, 29, 31, 32). In agreement with previous reports (29, 30, 32, 70, 71), GMSCs do not constitutively express IDO, but in response to IFN-γ stimulation, harbored a significantly increased level of functional IDO. Co-culture with GMSCs led to moderate suppression of mitogen-stimulated PBMC proliferation and IFN-γ secretion; however, the presence of stimulated PBMCs enhanced IL-10 secretion and IDO expression by GMSCs. Furthermore, the addition of IFN-γ neutralizing antibody significantly blocked the secretion of IL-10 and the expression of functional MO in GMSCs. These findings suggest that the up-regulated inflammatory signals dominated by IFN-γ in the co-culture of GMSCs and stimulated PBMCs can induce GMSC-mediated immunosuppression, mediated in part, via the up-regulation of IL-10 and functional IDO expression. However, further studies are required to determine whether other inflammatory cytokines such as TNF-γ and IL-16 are involved in priming GMSC-mediated immunosuppression.

Recently, several studies have reported that treatment with human bone marrow- or adipose-derived MSCs exhibits early efficacy in attenuating the progression of several experimental inflammatory diseases in murine models, including experimental arthritis (38), colitis (43, 46), and autoimmune encephalomyelitis (39). The apparent lack of graft rejection and positive treatment effects of human MSCs on these murine disease models could be due to their inherent capabilities to harness inflammatory cells infiltration, suppress inflammatory mediators production, and/or regulate immune tolerance by increasing the production of anti-inflammatory cytokines (e.g. IL-10) and inducing the generation/activation of Treg cells (38, 39, 43, 46). Most recently, a study by Gonzalez et al suggested that the viability of human adipose-derived MSCs was not required for their long-term immunosuppressive activities since these cells were only detectable in the recipient for about 1 week after injection (38). Similar to recent studies using hASC to treat experimental colitis (43, 46), the present invention has also demonstrated that infusion of GMSCs could ameliorate the severity of inflammatory-related colonic tissue injuries in experimental colitis without eliciting graft-versus-host disease response in immunocompetent animals. Not intending to be bound by any particular theory, we hypothesize that this may possibly be achieved by reducing colonic infiltrates of inflammatory cells, down-regulating the production of inflammatory cytokines, and by promoting the generation/activation of Treg cells.

Despite the potential benefits of MSCs in clinical applications, several questions remain unanswered, especially regarding the identity and biological properties of MSCs as compared to other stromal cells such as fibroblasts (72). Accumulating evidence has shown that MSCs share many common features with fibroblasts, including a spindle-like cell morphology, plastic adherence, expression profile of certain cell surface markers, multipotent differentiation and even immunomodulatory functions (72-74). Previous analysis of human bone marrow MSC subclones revealed that the lineage commitment was hierarchical in nature (75) and may differ among MSC subpopulations derived from different tissues (75, 76). As such, the so-called fibroblast population may represent a more differentiated subpopulation of MSCs (22, 76). Up to date, there is still a lack of evidence whether such hierarchy exists in relevance to several biological functions, specifically the immunomodulatory properties of MSCs, and should be further addressed.

In conclusion, the unique immunomodulatory and anti-inflammatory properties of GMSCs as well as their ease of isolation, abundant tissue source, and rapid ex vivo expansion render these post-natal stem cells an ideal source for stem cell-based therapeutic approaches in clinical applications, including inflammatory diseases.

### Reduction of Mucositis by GMSCs

Mucositis is the painful inflammation and ulceration of the mucous membranes lining the digestive tract, usually as an adverse effect of chemotherapy and radiotherapy treatment for cancer. Mucositis can affect up to 100% of patients undergoing high-dose chemotherapy and hematopoietic stem cell transplantation (HSCT), 80% of patients with malignancies of the head and neck receiving radiotherapy, and a wide range of patients receiving chemotherapy. For example, the commonly used anti-cancer agent, 5-fluorouracil (5-FU), leads to mucositis in up to about 40% of patients. Alimentary tract mucositis increases mortality and morbidity and contributes to rising health care costs. Unfortunately, currently available methods for treating mucositis are mostly supportive in nature.

In this example, we demonstrate that infusion of GMSCs is capable of reducing mucositis in 5-FU-induced alimentary tract mucositis in mouse models.

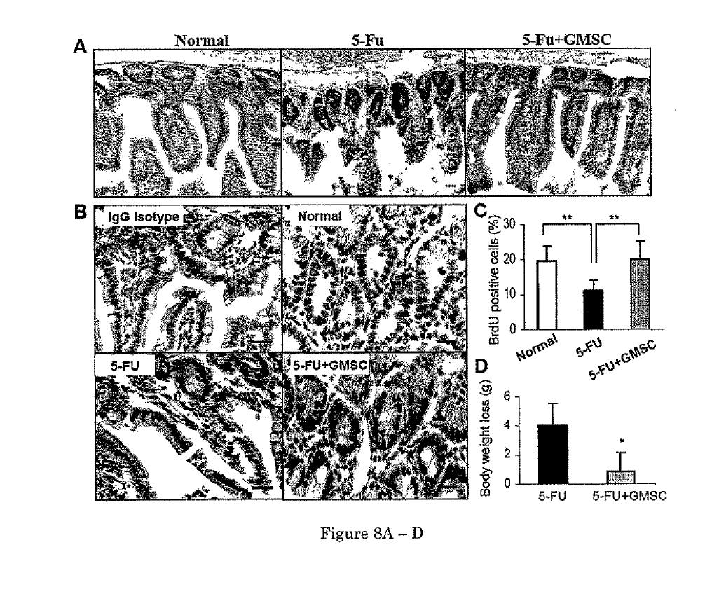

The mice were intraperitoneally (i.p.) injected with 5-FU (50 mg/kg body weight) for 4 consecutive days. One day after the last drug administration, one group of mice (n=5) were i.p. injected with 2×106 GMSCs. 6 days after cell injection, each mice was i.p. injected with 4 mg BrdU 2 hours before sacrifice. Then jejunum was collected, fixed in 10% formalin, and paraffin-embedded sections (spm) were cut for further analysis. FIG. 8 shows the result of the experiment.

FIG. 8A-B show histological comparisons of mice jejunum images before and after treatment.

As can be seen from FIG. 8C, BrdU immunohistochemical staining of the jejunum showed that 5-FU induced mucositic mice have drastically reduced BrdU positive cells, whereas GMSCs treated mice have nearly the same level of BrdU positive cells as normal mice.

FIG. 8D demonstrates that weight-loss in GMSCs treated mice have also significantly reduced compare to those in the 5-FU induced mucositic mice.

### Enhancement of Wound Healing by GMSCs

This example, demonstrates that wound healing is enhanced by infusion with GMSCs.

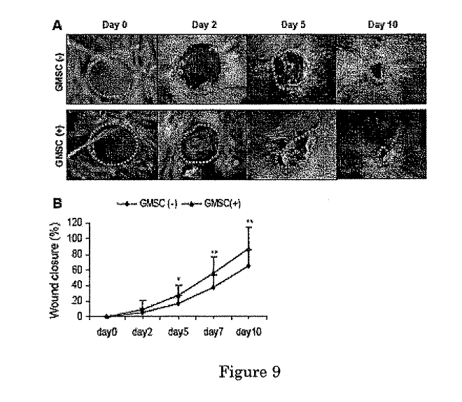

An wound was created by excision on the dorsal portion of C57BL/6 mice. One day after the wound, GMSCs (2×10⁶) were systemically infused by tail vein (i.v.) into mice and wound closure was daily observed. (FIG. 9A). Beginning at day 5, a significant increase in the rate of wound closure for the GMSC infused mice as compared to the control mice (FIG. 9B).

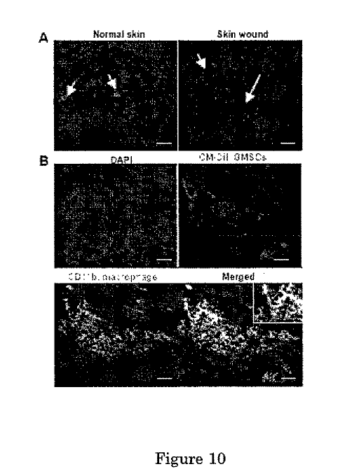

GMSCs prelabeled with CM-DiI were systemically infused by tail vein (i.v.) into mice one day after skin wounding. 7 days after cell injection, skin tissues were frozen sectioned and observed under a fluorescence microscope, whereby normal skin on the other side of the same mice were used as controls (FIG. 10A). Frozen sections of wounded skins from mice after injection with CM-DiI pre-labeled GMSCs were immunostained with FITC-conjugated antibody for mice CD11b (FIG. 10B). The results shows that GMSCs are home to the injury sites and interact with macrophages.

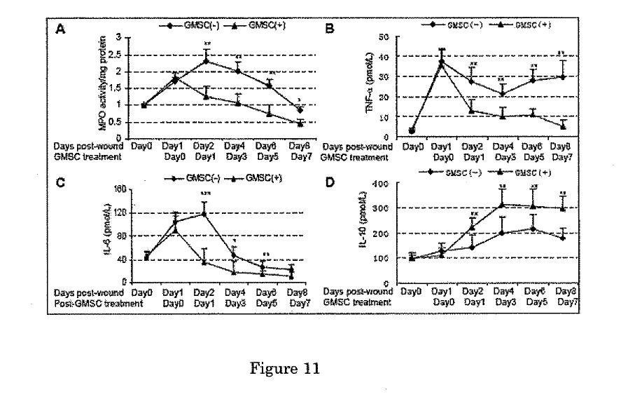

In order to show the anti-inflammatory abilities of GMSCs, a day after wound excision, GMSCs (2×10⁶) were injected via tail vein into mice. At different time points, injured skin was collected and tissue lysates were prepared for MPO activity assay (FIG. 11A) and ELISA assay on inflammatory cytokines, including TNF-α (FIG. 11B), IL-6 (FIG. 11C) and anti-inflammatory cytokine IL-10 (FIG. 11 D).

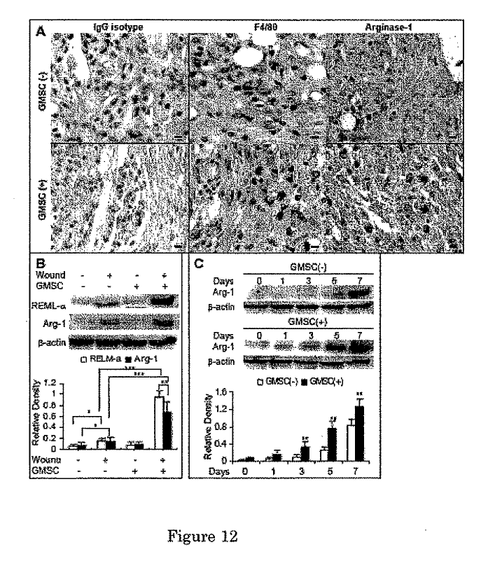

FIG. 12 shows that GMSCs promote the formation of alternatively activated macrophages (AAM) at the wounded sites. Paraffin-embedded sections of wounded skin after injection of GMSCs for 7 days were immunostained with specific antibodies for macrophages (F4/80) and alternatively activated macrophages (Arginase-1), showing increased numbers of AAM in the healed wound site after treatment with GMSCs as compared with no cell treatment (FIG. 12A). The increase in AAM formation in response to GMSC treatment was confirmed by Western blot analysis of arginase-1 (Arg-1) and RELM-α expression by AAM (FIG. 12B). GMSCs induced AAM formation in a time-dependent manner (FIG. 12C).

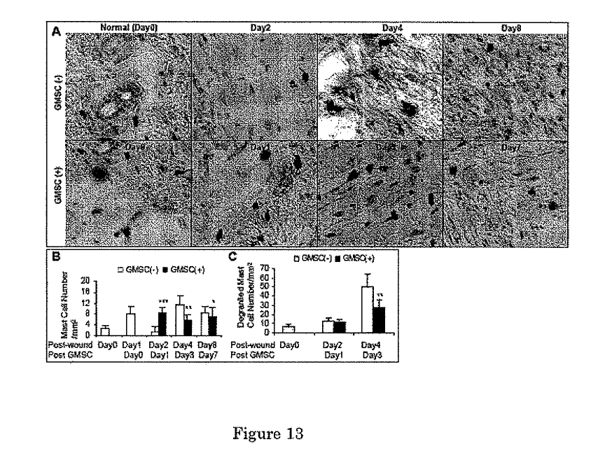

Finally the studies shows that GMSCs inhibit wound-stimulated degranuation of mast cells. promote the formation of alternatively activated macrophages (AAM) at the wounded sites. 24 hours after wound excision, GMSCs were injected via i.v. into mice. At different time points, wounded skin samples were collected and paraffin-embedded sections were prepared for toluidine staining of mast cells (FIG. 13A). Quantification of mast cell numbers (FIG. 13B). Quantification of degranuated or activated mast cells (FIG. 13C). The results showed that at day 2 post-injury, the number of mast cells significantly decreased in wound sites of mice without GMSC treatment, while GMSCs treatment maintained mast cells in the wound sites (FIGS. A and B). At day 4 post injury, the number of degranuated mast cells significantly increased while GMSC treatment led to a decrease in mast cell number (FIG. B) as well as stabilization of mast cells (FIGS. A and C).

Many modifications and variations of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

The entire contents of the following references are each incorporated herein by reference.
1. Prockop, D. J. 1997. Marrow stromal cells as stem cells for nonhematopoietic tissues. Science 276: 71-74.
2. Pittenger, M. F., A. M. Mackay, S. C. Beck, R. K. Jaiswal, R. Douglas, J. D. Mosca, M. A. Moorman, D. W. Simonetti, S. Craig, and D. R. Marshak. 1999. Multilieage potential of adult human mesenchymal stem cells. Science 284: 143-147.
3. Friedenstein, A. J., R. K. Chailakhjan, and K. S. Lalykina. 1970. The development of fibroblast colonies in monolayer cultures of guinea-pig bone marrow and spleen cells. Cell Tissue Kinet. 3: 393-403.
4. Fernandes, K. J., I. A. McKenzie, P. Mill, K. M. Smith, M. Akhavan, F. Barnabe-Heider, J. Biernaskie, A. Junek, N. R. Kobayashi, J. G. Toma, D. R. Kaplan, P. A. Labosky, V. Rafuse, C. C. Hui, and F. D. Miller. 2004. A dermal niche for multipotent adult skin-derived precursor cells. Nat. Cell
Biol. 6: 1082-1093.
5. Toma, J. G., I. A. McKenzie, D. Bagli, and F. D. Miller. 2005. Isolation and characterization of multipotent skin-derived precursors from human skin. Stem Cells 23: 727-737.
6. Kim, J. M., S. T. Lee, K. Chu, K. H. Jung, E. C. Song, S. J. Kim, D. I. Sinn, J. H. Kim, D. K. Park, K. M. Kang, N. Hyung Hong, H. K. Park, C. H. Won, K. H. Kim, M. Kim, S. Kun Lee, and J. K. Rob. 2007. Systemic transplantation of human adipose stem cells attenuated cerebral inflammation and degeneration in a hemorrhage stroke model. Brain Res. 1183: 43-50.
7. Bi, Y. M., D. Ehirchiou, T. M. Kilts, C. A. Inkson, M. C. Embree, W. Sonoyama, L. Li, A. I. Leet, B. M. Seo, L. Zhang, S. Shi, and M. F. Young. 2007. Identification of tendon stem/projenitor cells and the role of the extracellular matrix in their niche. Nat. Med. 13: 1219-1227.
8. Beltrami, A. P., D. Cesselli, N. Bergamin, P. Marcon, S. Rigo, E. Puppato, F. D'Aurizio, R. Verardo, S. Piazza, A. Pignatelli, A. Poz, U. Baccarani, D. Damiani, R. Fanin, L. Mariuzzi, N. Finato, P. Masolini, S. Burelli, O. Belluzzi, C. Schneider, and C. A. Beltrami. 2007. Multipotent cells can be generated in vitro from several adult human organs (heart, liver, and bone marrow). Blood 110: 3438-3446.
9. Chang, C. J., M. L. Yen, Y. C. Chen, C. C. Chien, H. I. Huang, C. H. Bai, and B. L. Yen. 2006. Placenta-derived multipotent cells exhibit immunosuppressive properties that are enhanced in the presence of interferon-□. Stem Cells 24: 2466-2477.
10. In't Anker, P. S., S. A. Scherjon, C. Kleijiburg-van der Keur, W. A. Noort, F. H. Claas, R. Willemze, W. E. Fibbe, and H. H. Kanhai. 2003. Amniotic fluid as a novel source of mesenchymal stem cells for therapeutic transplantation. Blood 102: 1548-1549.
11. Oh, W., D. S. Kim, Y. S. Yang, and J. K. Lee. 2008. Immunological properties of umbilical cord blood-derived mesenchymal stromal cells. Cell. Immunol. 251: 116-123.
12. Morsczeck, C., G. Schmalz, T. E. Reichert, F. Vollmer, K. Galler, and O. Driemel. 2008. Somatic stem cells for regenerative dentistry. Clin. Oral. Invest. 12: 113-118.
13. Gronthos, S., M. Mankani, J. Brahim, P. G. Robey, and S. Shi. 2000. Postnatal human dental pulp stem cells (DPSCs) in vitro and in vivo. Proc. Natl. Acad. Sci. USA 97: 13625-13630.
14. Gronthos, S., J. Brahim, W. Li, L. W. Fisher, N. Cheman, A. Boyde, P. DenBesten, P. G. Robey, and S. Shi. 2002. Stem cell properties of human dental pulp stem cells. J. Dent. Res. 81: 531-535.
15. Miura, M., S. Gronthos, M. Zhao, B. Lu, L. W. Fisher, P. G. Robey, and S. Shi. 2003. SHED: stem cells from human exfoliated deciduous teeth. Proc. Natl. Acad. Sci. USA 100: 5807-5812.
16. Seo, B. M., M. Miura, S. Gronthos, P. M. Bartold, S. Batouli, J. Brahim, M. Young, P. G. Robey, C. Y. Wang, and S. Shi. 2004. Investigation of multipotent postnatal stem cells from human periodontal ligament. Lancet 364: 149-155.
17. Morsczeck, C., W. Gotz, J. Schierholz, F. Zeilhofer, U. Kuhn, C. Mail, C. Sippel, and K. H. Hoffmann. 2005. Isolation of precursor cells (PCs) from human dental follicle of wisdom teeth. Matrix Biol. 24:155-165.
18. Lindroos, B., K. Mäenpää, T. Ylikomi, H. Oja, R. Suuronen, and S. Miettinen. 2009. Characterisation of human dental stem cells and buccal mucosa fibroblasts. Biochem. Biophys. Res. Commun. 368: 329-335.
19. Jo, Y. Y., H. J. Lee, S. Y. Kook, H. W. Choung, J. Y. Park, J. H. Chung, Y. H. Choung, E. S. Kim, H. C. Yang, and P. H. Choung. 2007. Isolation and characterization of postnatal stem cells from human dental tissues. Tissue Eng. 13: 767-773.
20. Gang, E. J., D. Bosnakovski, C. A. Figueiredo, J. W. Visser, and R. C. Perlingeiro. 2007. SSEA-4 identifies mesenchymal stem cells from bone marrow. Blood 109: 1743-1751.
21. Greco, S. J., K. Liu, and P. Rameshwar. 2007. Functional similarities among genes regulated by OCT4 in human mesenchymal and embryonic stem cells. Stem Cells 25: 3143-3154.
22. Covas, D. T., R. A. Panepucci, A. M. Fontes, W. A. Jr. Silva, M. D. Oreliana, M. C. Freitas, L. Neder, A. R. Santos, L. C. Peres, M. C. Jamur, and M. A. Zago. 2008. Multipotent mesenchymal stromal cells obtained from diverse human tissues share functional properties and gene-expression profiles with CD146+ perivascular cells and fibroblasts. Exp. Hematol. 36: 642-654.
23. Spaeth, E., A. Klopp, J. Dembinski, M. Andreeff, and F. Marini. 2008. Inflammation and tumor microenvironments: defining the migratory itinerary of mesenchymal stem cells. Gene Ther. 15: 730-738.
24. Karp, J. M., and G. S. Leng Teo. 2009. Mesenchymal stem cell homing: the devil is in the details. Cell Stem Cell 4: 206-216,
25. Nauta, A. J., and W. E. Fibbe. 2007. Immunomodulatory properties of mesenchymal stromal cells. Blood 110: 3499-3506.
26. Abdallah, B. M., and M. Kassem. 2009. The use of mesenchymal (skeletal) stem cells for treatment of degenerative diseases: current status and future perspectives. J. Cell. Physiol. 218: 9-12.
27. Uccelli, A., L. Moretta, and V. Pistoia. 2008. Mesenchymal stem cells in health and disease. Nat. Rev. Immunol. 8: 726-736.
28. Selmani, Z., A. Naji, I. Zidi, B. Favier, E. Gaiffe, L. Obert, C. Borg, P. Saas, P. Tiberghien, N. Rouas-Freiss, E. D. Carosella, and F. Deschaseaux. 2008. Human leukocyte antigen-G5 secretion by human mesenchymal stem cells is required to suppress T lymphocyte and natural killer function and to induce CD4+CD25highFoxP3+ regulatory T cells. Stem Cells 26: 212-222.
29. Aggarwal, S., and M. F. Pittenger. 2005. Human mesenchymal stem cells modulate allogeneic immune cell responses. Blood 105: 1815-1822.
30. Ryan, J. M., F. Barry, J. M. Murphy, and B. P. Mahon. 2007. Interferon-gamma does not break, but promotes the immunosuppressive capacity of adult human mesenchymal stem cells. Clin. Exp. Immunol. 149: 353-363.
31. Krampera, M, L. Cosmi, R. Angell, A. Pasini, F. Liotta, A. Andreini, V. Santarlasci, B. Mazzinghi, G. Pizzolo, F. Vinante, P. Romagnani, E. Maggi, S. Romagnani, and F. Annunziato. 2006. Role for Interferon—in the Immunomodulatory Activity of Human Bone Marrow Mesenchymal Stem Cells. Stem Cells 24: 386-398.
32. Delarosa, 0., E. Lombardo, A. Beraza, P. Mancheño, C. Ramírez, R. Menta, L. Rico, E. Camarillo, L. García, J. L. Abad, C. Trigueros, M. Delgado, and D. Büscher. 2009. Requirement of IFN-gamma-mediated indoleamine 2, 3 dioxygenase expression in the modulation of lymphocyte proliferation by human adipose-derived stem cells. Tissue Eng. Part A. [Epub ahead of print]
33. Spaggiari, G. M., A. Capobianco, H. Abdelrazik, F. Becchetti, M. C. Mingari, and L. Moretta. 2008. Mesenchymal stem cells inhibit natural killer-cell proliferation, cytotoxicity, and cytokine production: role of indoleamine 2,3-dioxygenase and prostaglandin E2. Blood 111: 1327-1333.
34. Sato, K., K. Ozaki, I. Oh, Meguro A, K. Hatanaka, T. Nagai, and K. Muroi. 2007. Ozawa. Nitric oxide plays a critical role in suppression of T-cell proliferation by mesenchymal stem cells. Blood 109: 228-234.
35. Polchert, D., J. Sobinsky, G. W. Douglas, M. Kidd, A. Moadsiri, E. Reina, K. Genrich, S. Mehrotra, S. Setty, B. Smith, and A. Bartholomew. 2008. IFN-γ activation of mesenchymal stem cells for treatment and prevention of graft versus host disease. Eur. J. Immunol. 38: 1745-1755.
36. Le Blanc, K., I. Rasmusson, B. Sundberg, C. Götherstöom, M. Hassan, M. Uzunel, and O. Ringdén. 2004. Treatment of severe acute graft-versus-host disease with third party haploidentical mesenchymal stem cells. Lancet 363: 1439-1441.
37. Lee, R. H., M. J. Seo, R. L. Reger, J. L. Spees, A. A. Pulin, S. D. Olson, and D. J. Prockop. 2006. Multipotent stromal cells from human marrow home to and promote repair of pancreatic islets and renal glomeruli in diabetic NOD/scid mice. Proc. Natl. Acad. Sci. USA 103:
17438-17443.
38. González, M. A., E. Gonzalez-Rey, L. Rico, D. Büscher, and M. Delgado. 2009. Treatment of experimental arthritis by inducing immune tolerance with human adipose-derived mesenchymal stem cells. Arthritis Rheum. 60: 1006-1019.
39. Zhang J, Li Y, Chen J, Cui Y, Lu M, Elias S B, Mitchell J B, Hammill L, Vanguri P, and Chopp M. 2005. Human bone marrow stromal cell treatment improves neurological functional recovery in EAE mice. Exp. Neurol. 195: 16-26.
40. Parekkadan, B., A. W. Tilles, and M. L. Yarmush. 2008. Bone marrow-derived mesenchymal stem cells ameliorate autoimmune enteropathy independent of regulatory T cells. Stem Cells 26:19134919.
41. Zhou, K., H. Zhang, O. Jin, X. Feng, G. Yao, Y. Hou, and L. Sun. 2008. Transplantation of human bone marrow mesenchymal stem cell ameliorates the autoimmune pathogenesis in MRL/lpr mice. Cell. Mol. Immunol. 5: 417-424.
42. Liu, Y., Y. Zheng, G. Ding, D. Fang, C. Zhang, P. M. Bartold, S.

Gronthos, S. Shi, and S. Wang. 2008. Periodontal ligament stem cell-mediated treatment for periodontitis in miniature swine. Stem Cells 26: 1065-1073.
43. González, M. A., E. Gonzalez-Rey, L. Rico, D. Bascher, and M. Delgado. 2009. Adipose-derived mesenchymal stem cells alleviate experimental colitis by inhibiting inflammatory and autoimmune responses. Gastroenterology 136: 978-989.
44. Németh, K., A. Leelahavanichkul, P. S. Yuen, B. Mayer, A. Parmelee, K. Doi, P. G. Robey, K. Leelahavanichkul, B. H. Koller, J. M. Brown, X. Hu, I. Jelinek, R. A. Star, and E. Mezey. 2009. Bone marrow stromal cells attenuate sepsis via prostaglandin E (2)-dependent reprogramming of host macrophages to increase their interleukin-10 production. Nat. Med. 15: 42-49.
45. Iyer, S. S., and M. Rojas. 2008. Anti-inflammatory effects of mesenchymal stem cells: novel concept for future therapies. Exp. Opion Biol. Ther. 8: 569-581.
46. Gonzalez-Rey, E., P. Anderson, M. A. González, L. Rico, D. Büscher, and M. Delgado. 2009. Human adult stem cells derived from adipose tissue protect against experimental colitis and sepsis. GUT 58: 929-939.
47. Irwin, C. R., M. Picardo, I. Ellis, P. Sloan, A. Grey, M. McGurk, and S. L. Schor. 1994. Inter- and intra-site heterogeneity in the expression of fetal-like phenotypic characteristics by gingival fibroblasts: potential significance for wound healing. J. Cell Sci. 107 (Pt5): 1333-1346.
48. Stephens, P., K. J. Davies, N. Occleston, R. D. Pleass, C. Kon, J. Daniels, P. T. Khaw, and D. W. Thomas. 2001. Skin and oral fibroblasts exhibit phenotypic differences in extracellualr matrix reorganization and matrix metalloproteinase activity. Br. J. Dermatol. 144: 229-237,
49. Novak, N., J. Haberstock, T. Bieber, and J. P. 2008. Allam The immune privilege of the oral mucosa. Trends Mol. Med. 14: 191-198.
50. Jones, P. H., and F. M. Watt. 1993. Separation of human epidermal stem cells from transit amplifying cells on the basis of differences in integrin function and expression. Cell 73: 713-724.
51. Podolsky, D. K. 2002. Inflammatory bowel disease. N. Engl. J. Med. 347: 417-429.
52. Xavier, R. J., and D. K. Podolsky. 2007. Unravelling the pathogenesis of inflammatory bowel disease, Nature 448: 427-434.
53. Mizoguchi, A., and E. Mizoguchi, 2008. Inflammatory bowel disease, past, present and future: lessons from animal models. J. Gastroenterol. 43: 1-17,
54. Alex, P., N. C. Zachos, T. Nguyen, L. Gonzales, T. E. Chen, L. S. Conklin, M. Centola, and X. Li. 2009. Distinct cytokine patterns identified from multiplex profiles of murine DSS and TNBS-induced colitis. Inflamm. Bowel. Dis. 15: 341-352.
55. Shi, S., S. Gronthos, S. Chen, A. Reddi, C. M. Counter, P. G. Robey, and C. Y. Wang. 2002. Bone formation by human postnatal bone marrow stromal stem cells is enhanced by telomerase expression. Nat. Biotech. 20: 587-591.
56. Yamaza, T., Y. Miura, Y. Bi, Y. Z. Liu, K. Akiyama, W. Sonoyama, V. Patel, S. Gutkind, M. Young, S. Gronthos, A. Le, C-Y. Wang, W. J. Chen, and S. Shi. 2008. Pharmacologic stem cell based intervention as a new approach to osteoporosis treatment in rodents. PlosOne 3: e2615
57. Bartsch, G., J. J. Yoo, P. De Coppi, M. M. Siddiqui, G. Schuch, H. G. Pohl, J. Fuhr, L. Perin, S. Soke, and A. Atala. 2005. Propagation, expansion, and multilineage differentiation of human somatic stem cells from dermal progenitors. Stem Cell Dev. 14: 337-348.
58. You, S., J. H. Moon, T. K. Kim, S. C. Kim, J. W. Kim, D. H. Yoon, S. Kwak, K. C. Hong, Y. J. Choi, and H. Kim. 2004. Cellular characteristics of primary and immortal canine embryonic fibroblast cells. Exp. Mal. Med. 36: 325-335.
59. Gregory, C. A., W. G. Gunn, A. Peister, and D. J. Prockop. 2004. An Alizarin red-based assay of mineralization by adherent cells in culture: comparison with cetylpyridinium chloride extraction. Anal. Biochem. 329: 77-84.
60. Yu, W., Z. Chen, J. Zhang, L. Zhang, H. Ke, L. Huang, Y. Peng, X. Zhang, S. Li, B. T. Lahn, and A. P. Xiang. 2007. Critical role of phosphoinositide 3-kinase cascade in adipogenesis of human mesenchymal stem cells. Mol. Cell. Biochem. 310: 11-18.
61. Tao, H., R. Rao, and D. Ma. 2005. Cytokine-induced stable neuronal differentiation of human bone marrow mesenchymal stem cells in a serum/feeder cell-free condition. Dev. Growth Differ. 47: 423-433.
62. Cipriani, P., S. Guiducci, I. Miniati, M. Cinelli, S. Urbani, A. Marrelli, V. Dolo, A. Pavan, R. Saccardi, A. Tyndall, R. Giacomelli, and M. M. Cerinic. 2007. Impairment of endothelial cell differentiation from bone marrow-derived mesenchymal stem cells: new insight into the pathogenesis of systemic sclerosis. Arthritis Rheum. 56: 1994-2004.
63. Morsczeck, C., G. Schmalz, T. E. Reichert, F. \Tanner, K. Galler, and O. Driemel. 2008. Somatic stem cells for regenerative dentistry. Clin. Oral Invest. 12: 113-118.
64. Cournil-Henrionnet, C., C. Huselstein, Y. Wang, L. Galois, D. Mainard, V. Decot, P. Netter, J. F. Stoltz, S. Muller, P. Gillet, and A. Watrin-Pinzano. 2008. Phenotypic analysis of cell surface markers and gene expression of human mesenchymal stem cells and chondrocytes during monolayer expansion. Biorheology. 45: 513-526.
65. Lorenz , K., M. Sicker, E. E. Schmelzer, T. Rupf, J. Salvetter, M. Schulz-Siegmund, and A. Bader. 2008. Multilineage differentiation potential of human dermal skin-derived fibroblasts. Exp. Dermatol. 17: 925-932.
66. Di Nicola, M., C. Carlo-Stella, M. Magni, M. Milanesi, P. D. Longoni, P. Matteucci, S. Grisanti, and A. M. Gianni. 2002. Human bone marrow stromal cells suppress T-lymphocyte proliferation induced by cellular or nonspecific mitogenic stimuli. Blood 99: 3838-3843.
67. Cui, L., S. Yin, W. Liu, N. Li, W. Zhang, and Y. Cao. 2007. Expanded adipose-derived stem cells suppress mixed lymphocyte reaction by secretion of prostaglandin E2. Tissue Eng. 13: 1185-1195.
68. Wang, M., Y. Yang, D. Yang, F. Luo, W. Liang, S. Guo, and J. Xu. 2009. The immunomodulatory activity of human umbilical cord blood-derived mesenchymal stem cells in vitro. Immunology 126:220-232.
69. Sheng, H., Y. Wang, Y. Jin, Q. Zhang, Y. Zhang, L. Wang, B. Shen, S. Yin, W. Liu, L. Cui, and N. Li. 2008. A critical role of IFN gamma in priming MSC-mediated suppression of T cell proliferation through up-regulation of B7-H1. Cell Res. 18: 846-857.
70. Wada, N., D. Menicanin, S. Shi, P. M. Bartold, and S. Gronthos. 2009. Immunomodulatory properties of human periodontal ligament stem cells. J. Cell. Physiol. 219: 667-676.
71. Mahanonda, R., N. Sa-Ard-Iam, P. Montreekachon, A. Pimkhaokham, K. Yongvanichit, M. M. Fukuda, and S. Pichyangkul. 2007. IL-8 and IDO expression by human gingival fibroblasts via TLRs. J. Immunol. 178: 1151-1157.
72. Haniffa, M. A., M. P., Collin, C. D., Buckley, and F. Dazzi. 2009. Mesenchymal stem cells: the fibroblasts' new clothes? Haematologica 94: 258-263.
73. Lysy, P. A., F. Smets, C. Sibille, M. Najimi, and E. M. Sokal. 2007. Human skin fibroblasts: From mesodermal to hepatocyte-like differentiation. Hepatology 46: 1574-1585.
74. Haniffa, M. A., X. N. Wang, U. Holtick, M. Rae, J. D. Isaacs, A. M. Dickinson, C. M. Hilkens, and M. P. Collin. 2007. Adult human fibroblasts are potent immunoregulatory cells and functionally equivalent to mesenchymal stem cells. J. Immunol. 179: 1595-1604.
75. Muraglia, A., R. Cancedda, and R. Quarto. 2000. Clonal mesenchymal progenitors from human bone marrow differentiate in vitro according to a hierarchical model. J. Cell Sci. 113: 1161-1166.
76. Sudo, K., M. Kanno, K. Miharada, S. Ogawa, T. Hiroyama, K. Saijo, and Y. Nakamura. 2007. Mesenchymal progenitors able to differentiate into osteogenic, chondrogenic, and/or adipogenic cells in vitro are present in most primary fibroblast-like cell populations. Stem Cells 25:1610-1617.

What is claimed is:

1. Isolated gingiva derived messenchymal stem cells (GMSCs).

2. The isolated GMSCs according to claim 1, wherein the cells are capable of clonogenicity, multiple differentiation capacity, and self-renewal.

3. The isolated GMSCs according to claim 2, wherein the GMSCs are capable of multiple differentiation into adipocytes, neural cells, endothelial cells, or osteoblasts.

4. A composition comprising isolated GMSCs according to claim 1.

5. A method of isolating GMSCs, comprising obtaining gingival tissue, treating the tissue with dispase to allow for separation into an epithelial and spinous layers, mincing the tissue, digesting the tissue with collagenase, filtering and collecting digested cells, plating the cells and allowing the cells to grow.

6. A method of treating an inflammatory and/or autoimmune disease in a subject, comprising:
a) administering GMSCs into the subject;
b) comparing the amount of inflammation at the affected organ or site in a control subject with the treated subject; and
c) determining that the amount of inflammation in the subject given GMSCs is less than the amount of inflammation in the control subject is indicative of treating the inflammatory and/or autoimmune disease.

7. The method according to claim 6, wherein the inflammatory response is normal or pathological wound healing, the inflammatory and/or autoimmune disease is graft-versus-host disease (GvHD), diabetes, rheumatoid arthritis (RA), autoimmune encephalomyelitis, systemic lupus erythematosus (SLE), multiple sclerosis (MS), periodontitis, intestinal and bowel disease (IBD), alimentary tract mucositis induced by chemo- or radiotherapy, or sepsis.
